(19) United States
(12) Patent Application Publication
(10) Pub. No.: US 2020/0079860 A1
(43) Pub. Date: Mar. 12, 2020

(54) METHODS AND COMPOSITIONS FOR TUMOR THERAPY

(71) Applicant: MEMORIAL SLOAN KETTERING CANCER CENTER, New York, NY (US)

(72) Inventors: Danny Nejad Khalil, New York, NY (US); Jedd D. Wolchok, New York, NY (US); Taha Merghoub, Jersey City, NJ (US)

(21) Appl. No.: 15/750,496

(22) PCT Filed: Aug. 8, 2016
(86) PCT No.: PCT/US16/45970

§ 371 Date: Feb. 5, 2018

Related U.S. Application Data
(60) Provisional application No. 62/202,163, filed on Aug. 6, 2015.
(60) Provisional application No. 62/287,407, filed on Jan. 26, 2016.

(51) Int. Cl. C07K 16/28; A61P 35/00; A61K 39/395; C07K 16/24; A61K 31/7032; A61K 31/7088; A61K 47/68; A61K 47/69

(52) U.S. Cl. CPC C07K 16/2878; C07K 16/2896; A61P 35/00; A61K 39/3955; C07K 16/244; A61K 2039/507; A61K 31/7032; A61K 31/7088; A61K 47/6849; A61K 47/6929; C07K 16/2866

(57) ABSTRACT

The present invention provides various compositions and methods useful for the treatment of cancer, such as cancers that are resistant to immune checkpoint blockade and/or are resistant to treatment with PD-1, PD-L1 or CTLA-4 inhibitors. In some embodiments the present invention provides compositions comprising one or more CD40 agonists (e.g. CD40 agonist antibodies), TLR agonists, and/or IL10 receptor inhibitors or IL10 inhibitors, and/or various combinations thereof, optionally together with one or more immune checkpoint inhibitors, and the use of such compositions in treatment of tumors.

### CROSS-REFERENCE TO RELATED APPLICATIONS

This application claims the benefit of priority of U.S. Provisional Patent Application No. 62/202,163 filed on Aug. 6, 2015, and U.S. Provisional Patent Application No. 62/287,407 filed on Jan. 26, 2016, the contents of each of which are hereby incorporated by reference in their entireties.

### INCORPORATION BY REFERENCE

For the purpose of only those jurisdictions that permit incorporation by reference, all of the references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention.

### BACKGROUND

Immune checkpoint blockade (ICB) is an approach to treating cancer that involves blocking inhibitory immune-cell receptors, such as PD-1, PD-L1, and/or CTLA-4, present on T-cells. Several such immune checkpoint inhibitors are currently in use clinically—including pembrolizumab, nivolumab, atezolizumab, and ipilimumab. While such methods can lead to durable and occasionally complete tumor regression in some patients, other patients remain insensitive to such treatments. For example, response rates to anti-PD-1 monotherapy range from approximately 44% in melanoma patients to markedly lower rates in breast and colorectal cancer patients. Accordingly, there is a need in the art for new and improved treatment regimens that can be used to treat tumors in that subset of patients for which immune checkpoint inhibitors are not effective.

### SUMMARY OF THE INVENTION

The present invention is based, in part, on a series of important discoveries that are described in more detail in the Examples section of this patent specification. For example, it has now been discovered that certain combinations of agents, such as CD40 agonists and TLR agonists, can be used to treat tumors. Furthermore, it has been found that such combinations of agents can be used to sensitize tumor cells to treatment with immune checkpoint inhibitors, such as PD-1, PD-L1, and/or CTLA-4 inhibitors, leading to complete tumor regression, even in tumors that were previously resistant to such treatments. Building on these discoveries, and other discoveries presented herein, the present invention provides a variety of new and improved compositions and methods for the treatment of tumors. Some of the main aspects of the present invention are summarized below. Additional aspects of the invention are provided and described in the Detailed Description, Drawings, Examples, and Claims sections of this patent application.

In some embodiments the present invention provides a method of treating a tumor in a subject in need thereof, the method comprising administering to the subject an effective amount of: (a) a CD40 agonist (such as a CD40 agonist antibody) and (b) a TLR agonist. Similarly, in some embodiments the present invention provides a method of treating a tumor in a subject in need thereof, comprising administering to the subject an effective amount of: (a) a CD40 agonist (such as a CD40 agonist antibody), (b) a TLR agonist and (c) an immune checkpoint inhibitor (such as a PD-1, PD-L1, or anti-CTLA-4 inhibitor). Furthermore, each of the above embodiments may also comprise administering to the subject an effective amount of an IL10 receptor-blocking antibody or an IL10-blocking antibody. Similarly, each of the above embodiments may also comprise administering to the subject an effective amount of a vaccine adjuvant, or a vaccine antigen.

In each of the treatment methods of the present invention the various different active agents, or combinations thereof, can be administered either systemically or locally or a combination of both. Suitable routes of local administration include, but are not limited to, intratumoral, intrahepatic, intrapleural, intraocular, intraperitoneal, and intrathecal administration.

In some preferred embodiments the CD40 agonist (e.g. CD40 agonist antibody), the TLR agonist, and/or the IL10 receptor-blocking antibody or IL10 blocking antibody is administered locally, such as intratumorally. However, in other embodiments the CD40 agonist, the TLR agonist, and/or the IL10 receptor-blocking antibody/IL10-blocking antibody is administered systemically.

In some preferred embodiments the immune checkpoint inhibitor (such as an anti-PD-1, anti-PD-L1, or anti-CTLA-4 agent) is administered systemically. However, in other embodiments the immune checkpoint inhibitor is administered locally, such as intratumorally.

In some such embodiments the subject has a tumor that is resistant to treatment with an immune checkpoint inhibitor. In some such embodiments the subject has a PD-1, PD-L1, and/or CTLA-4 inhibitor resistant tumor. In some such embodiments the subject has previously been treated with an immune checkpoint inhibitor (such as a PD-1, PD-L1, or CTLA-4 inhibitor). In some such embodiments that patient has not previously been treated (with immunotherapy, checkpoint blockade, or otherwise). In some such embodiments the tumor is any solid tumor, including, but not limited to, a melanoma, a breast tumor, a lung tumor (such as a small cell lung cancer tumor), a prostate tumor, an ovarian tumor, a sarcoma, and a colon tumor.

In some embodiments the present invention provides various compositions, such as pharmaceutical compositions, that may be useful in the above methods. For example, in some embodiments the present invention provides compositions, such as pharmaceutical compositions, comprising: (a) a CD40 agonist (such as a CD40 agonist antibody), and (b) a TLR agonist, or compositions comprising any other combination of the active agents described (i.e. CD40 agonists, TLR agonists, IL10 receptor blocking antibodies/IL10 blocking antibodies, or immune checkpoint inhibitors (such as PD-1, PD-L1, and/or CTLA-4 inhibitors). In some such embodiments the compositions also comprise a vaccine adjuvant, or a vaccine antigen.

In some such embodiments the CD40 agonist (e.g. CD40 agonist antibody) and the TLR agonist, or any one or more of the active agents described above (i.e. CD40 agonists, TLR agonists, IL10 receptor or IL10 blocking antibodies, or immune checkpoint inhibitors), are connected via a linker moiety to form a single molecule, such as an antibody-drug conjugate molecule. In some such embodiments the agents may be connected using a lysine-bound linker or a cysteine-bound linker.

In some such embodiments any one or more of the active agents described above (i.e. CD40 agonists, TLR agonists, IL10 receptor blocking antibodies, or immune checkpoint inhibitors) may be provided together using a nanoparticle. For example, in some embodiments the CD40 agonist (e.g. CD40 agonist antibody) and the TLR agonist are provided together in a nanoparticle. Similarly in some embodiments the CD40 agonist (e.g. CD40 agonist antibody), the IL10 receptor blocking antibody, and the TLR agonist are provided together in a nanoparticle. In some such embodiments the CD40 agonist (e.g. CD40 agonist antibody) and/or the IL10 receptor-blocking antibody (or IL10-blocking antibody) is present on the surface of the nanoparticles. In particular it has been found that the nanoparticles of the invention are particularly effective when an IL10 receptor-blocking antibody is provided on the surface of the nanoparticles (e.g. in addition to a CD40 agonist antibody). However, in other embodiments these agents can be included inside nanoparticles—as cargo. In some such embodiments the TLR agonist and/or the immune checkpoint inhibitor (such as PD-1, PD-L1, and/or CTLA-4 inhibitor) is present inside the nanoparticles—i.e. as the “cargo” within the nanoparticle. In particular it has been found that the nanoparticles of the invention are particularly effective when the TLR3 agonist polyIC is provided as “cargo” within the nanoparticles. However, in other embodiments these agents can be used on the surface of the nanoparticles. The nanoparticles of the present invention can comprise the various active agents in any location—i.e. either coated on the surface of the nanoparticles or inside the nanoparticles.

In some such embodiments the nanoparticle is made using any suitable nanoparticle chemistry or technology known in the art. In some such embodiments the nanoparticle comprises one or more agents selected from the group consisting of mannose, chitosan, manosylated chitosan, protamine, chitosan with protamine, albumin, PLGA, and fucoidan. In some such embodiments the nanoparticles are formulated to release the active agent within them (i.e. their cargo) at endosomal pH, for example at the pH of early endosomes. The pH sensitivity of the nanoparticles can be adjusted (e.g., by adjusting their density) so the nanoparticles can be made to degrade within the acidic endosomes of APCs. In some the chemical features or physical properties (e.g., size, charge, etc) of the nanoparticles can be controlled such that systemic administration will lead to enrichment of the nanoparticles in certain organs of interest (e.g., the liver in the case of tumors within the liver or the lung in the case of tumors within the lungs). Means for altering the chemical or physical properties of nanoparticles to allow for tissue-specific enrichment are known in the art and can be used in connection with the present invention. For example, it is known that galactosamine-modified polymers can be used to target asiolaglycoprotein-receptor overexpressed by liver cells as a means for targeted delivery to the liver. See Seymour et al., “Hepatic drug targeting: phase I evaluation of polymer-bound doxorubicin,” J. Clin. Oncol. 2002, Vol. 20(6), pp. 1668-76, the contents of which are hereby incorporated by reference.

In those embodiments where nanoparticles are used to deliver the active agents of the invention, it has been found the nanoparticle compositions may be delivered using any suitable route of administration—whether local or systemic. However, in preferred embodiments intravenous administration is used. In particular, it has been found that the nanoparticle compositions of the invention are particularly potent when administered intravenously, such that the nanoparticles can be administered intravenously at approximately the same (low) dose with which they are administered intratumorally.

In some embodiments the CD40 agonist used in the methods and compositions described herein is selected from the group consisting of the following antibodies: FGK45, CP-870,984, APX005M, dacetuzumab, ChiLob 7/4, a CD40 agonist antibody as described in WO2005/063289, and a CD40 agonist antibody as described in WO2013/034904.

In some embodiments the TLR agonist used in the methods and compositions described herein is any TLR agonist known in the art that binds to a TLR expressed by an antigen presenting cell (APC), such as a dendritic cell (DC), macrophages, tissue-resident macrophages, monocytes, monocyte-derived cells, B-Cells, neutrophils, langerhans cells, histiocytes, or any so-called professional or non-professional APC. In some embodiments the TLR agonist is a TLR4 agonist, such as monophosphoryl lipid A (MPL). In some embodiments the TLR agonist is a TLR3 agonist, such as polyI:C.

In some embodiments the immune checkpoint inhibitor (including but not limited to PD-1, PD-L1, and/or CTLA-4 inhibitor) used in the methods and compositions described herein is an antibody. In some such embodiments the immune checkpoint inhibitor is an antibody selected from the group consisting of pembrolizumab, nivolumab, atezolizumab, ipilimumab, and the PD-1 inhibitor antibody RMP1-14.

In some embodiments the IL10 receptor blocking antibody used in the methods and compositions described herein is the antibody 1B1.3A.

These and other embodiments are further described in other sections of this patent application. Furthermore, one of skill in the art will recognize that the various embodiments of the present invention described can be combined in various different ways, and that such combinations are within the scope of the present invention.

### BRIEF DESCRIPTION OF THE DRAWINGS

FIG. 1. Schematic illustration of a treatment approach of the current invention, whereby immune resistant tumors are subjected to enforced APC activation. Activation of APCs at the tumor site, where they are continually exposed to tumor antigens, can induce the priming and expansion of tumor-specific T-cells. Such T-cells can then circulate and enter the tumor microenvironment where PD-1 blockade can free them to lyse tumor cells presenting cognate antigens.

FIG. 2. Schematic illustration of a treatment regimen used in performing experiments described in several of the Examples. By injecting only one of two tumors throughout the course of the experiment it is possible to separate the effect of the injected tumor from the “abscopal” effect on the distant non-injected tumor. Once treatment begins, tumors are measured twice weekly for at least 90 days.

FIG. 3. Tumor growth curves of “injected” and “non-injected” tumors in “control” and “treatment” groups—as further described in Example 1. In all experiments the treated mice were treated with MPL (intratumoral) at 5 μg, anti-CD40 (intratumoral) at 20 μg, and anti-PD-1 (systemically by intraperitoneal injection) at 250 μg, while control mice were treated with isotype control antibodies and vehicle only. Each line/curve represents measurements of tumor size from one individual tumor over time. (Time in days is indicated on the X axis. Tumor size in mm2 is indicated on the Y axis). Individual tumor growth curves demonstrate rapid cell-kill of the injected tumor followed by control or eradication of non-injected tumors.

FIGS. 4A-B. Data showing that animals re-implanted with tumors fully resist new tumor growth. FIG. 4A—Tumor growth curves of “naïve” (left panel) and “previously treated” (right panel) tumors—as further described in Example 1. Each line/curve represents measurements from one individual tumor over time. Previously-treated animals re-implanted with tumor cells all resist the development of tumors at 90 days, whereas control naïve animals all develop aggressive tumors. This indicates that anti-tumor immunologic memory is generated by the treatment regimen and is sufficiently robust to resist tumor re-challenge and prevent tumor recurrence. FIG. 4B—Photograph of mouse treated as described herein. Re-challenged animals develop fur-depigmentation both at the injected original site (right side of mouse) and at the site of the 90-day re-implanted tumor cells (left side of mouse), while surrounding tissue is unaffected. This is consistent with a highly specific anti-melanoma/melanocyte adaptive immune response that is developed during treatment, and that persists well after treatment has ended.

FIG. 5. Schematic illustration of MPL-CD40 agonist mAb nanoparticles. The nanometer-scale spheres are coated with anti-CD40 mAb and carry monophosphoryl lipid A (MPL) as their internal cargo. The anti-CD40 mAb serves to simultaneously target and activate myeloid cells. The MPL provides a second activation signal once the nanoparticle is internalized by the targeted myeloid cell. Myeloid cells include those that directly kill tumor cells, as well as APCs that prime T cells to kill tumor cells throughout the organism.

FIGS. 6A-B. Data showing that the nanoparticle formulation shown in FIG. 5 is superior to “non-formulated” mixtures of anti-CD40 mAb and MPL at identical concentrations. In all experiments mice in the “treatment” groups were treated with intratumoral MPL at 5 μg and intratumoral anti-CD40 at 20 μg (whether those agents were non-formulated or formulated as a nanoparticle), as well as intraperitoneal anti-PD-1 at 250 μg, while mice in the “control” groups were treated with isotype control antibodies and vehicle only. FIG. 6A—Individual tumor growth curves for “control,” “treatment (non-formulated),” and “treatment (formulated as nanoparticle)” treatment groups for both “injected” and “non-injected” tumors—as indicated in the figure. Each line/curve represents measurements of tumor size from one individual tumor over time. (Time in days is indicated on the X axis. Tumor size in mm² is indicated on the Y axis). FIG. 6B—Average tumor growth curves for “non-formulated” and “nanoparticle formulation” treatment groups. Results in both FIG. 6A and FIG. 6B are from animals treated with intraperitoneal anti-PD1 and intratumoral antiCD40 and MPL (in the two treatment groups). The nanoparticle treated group achieved complete eradication of all injected tumors, as compared to the non-formulated mixture. Results with chitosan nanoparticles are depicted here. Similar results were obtained with nanoparticles formulated with albumin, mannose, PLGA, fucoidan, and chitosan with protamine.

FIGS. 7A-7F. Data showing that, in addition to clearing the injected tumor, treatment with MPL, anti-CD40, and anti-PD-1 converts the treated tumor into a ‘cellular factory’ capable of priming anti-tumor T lymphocytes that subsequently infiltrate and attack distant non-injected tumors. In all experiments the treated mice were treated with MPL (intratumoral) at 5 μg, anti-CD40 (intratumoral) at 20 μg, and anti-PD-1 (systemic, via intraperitoneal injection) at 250 μg, while control mice were treated with isotype control antibodies and vehicle only. FIG. 7A Graph with data showing that treatment with MPL (intratumoral), anti-CD40 (intratumoral) and anti-PD-1 (systemic, via intraperitoneal injection) induces extensive neutrophil infiltration into injected tumors within 3 hr of treatment. The X axis shows time points after initial treatment ranging from 3 hours to 2 weeks. The Y axis shows the % of live CD45 cells that are CD11b+ and Ly6G+. FIG. 7B—Graph with data showing that neutrophils within injected tumors upregulate CD86 in response to treatment indicating that they are activated and able to prime T lymphocytes. The X axis shows time points after initial treatment ranging from 3 hours to 2 weeks. The Y axis shows the % of CD11b+/Ly6g+ cells that are CD86+. FIG. 7C—Graph with data showing that dendritic cells upregulate CD86 within injected tumors as they too are converted into activated APCs able to prime T lymphocytes. The X axis shows time points after initial treatment ranging from 3 hours to 1 week. The Y axis shows CD86 mean fluorescence intensity among CD11c+ cells. FIG. 7D—Microscope images showing that one week after initiating treatment lymphocytes infiltrate non-injected tumors in “treated,” but not “control”, animals. FIG. 7E—Flow cytometry data confirming that cytotoxic CD8 T lymphocytes infiltrate non-injected tumors. The X axis shows time points after initial treatment ranging from 3 hours to 2 weeks. The Y axis shows the % of CD8+ cells among live CD45+ cells. FIG. 7F—Data showing that cytotoxic CD8 T lymphocyte proliferation within non-injected tumors is enhanced by treatment also. The X axis shows time points after initial treatment ranging from 3 hours to 2 weeks. The Y axis shows the % of ki67+ cells among live, CD45+, CD8+ cells.

FIG. 8. Data showing that the impact of treatment is almost completely lost in animals lacking functional lymphocytes. Experiments were performed as for FIG. 3 with the exception that the data was generated using animals lacking functional lymphocytes (RAG-1 KO mice)—as further described in Example 1. Tumor growth curves for “control” (left-hand graphs) and “treatment” groups (right-hand graphs) in “injected” (top graphs) and non-injected (bottom graphs) tumors are shown. Each line/curve represents measurements from one individual tumor over time.

FIG. 9. Averaged tumor growth curves for “control” and “treatment” groups in both the “injected tumor” and “distant non-injected tumor”—with treatments as described in Example 2.

FIG. 10A-10C. Data showing immune cell populations 24 hours after one treatment with intratumoral MPL (5 μg), intratumoral anti-CD40 (20 μg), and intratumoral anti-IL10R (100 μg). Control data was obtained by treatment with isotype control antibodies and vehicle. FIG. 10A—Analysis of maturation markers on conventional DCs (cDCs) showing that CD86 remains elevated in the tumor at 24 hours. Y axes show mean fluorescence intensity among CD11b+, CD11c+ cells of CD86 (first column), CD80 (second column), and MHC-II (third column). FIG. 10B—Data showing that tumors show evidence of regulatory T cell depletion. The graph shows FoxP3-positive regulatory T cells (Tregs) as a percent of CD4 positive cells within the tumor. The “control” is isotype (non-specific) antibody control and vehicle. The “treatment” is intratumoral CD40 mAb, MPL, and IL10R mAb. There was no anti-PD-1 treatment. FIG. 10C—Data showing that CD4 T-cells in draining lymph nodes (DLNs) show enhanced expression of the cytolytic enzyme granzye B, and tumor necrosis factor α (TNF α) upon re-stimulation. The “treatment” and “control” are the same as in FIG. 10B. The Y axis shows mean fluorescence intensity (MFI) of granzyme B or TNF alpha among CD4 T cells in the DLNs. “NDLN” refers to non-draining lymph nodes.

FIG. 11A-11C. Data showing that addition of IL10 receptor blockage augments systemic potency. FIG. 11A. Averaged tumor growth curves for the “injected tumor” and “distant non-injected tumor” in “control” and “treatment” groups—as detailed in Example 2. In addition to eradication of injected tumors, 80% of non-injected tumors also exhibited complete regression. Survival graph of animals described in 11A are presented in FIG. 11B. FIG. 11C shows a treated mouse exhibiting fur depigmentation at the site of an eradicated tumor.

FIG. 12A-12B. Tumor growth curves of “injected tumors” and “contralateral tumors” (i.e. non-injected tumors in “control tx” treatment and “triple tx” treatment” groups—as further described in Example 2. Each line/curve represents measurements from one individual tumor over time. Triple agent treatment (“triple tx”) consisted of treatment with a combination of intratumoral (IT) MPL, IT CD40 agonist mAb, and IT IL10R blocking mAb. FIG. 12A provides growth curves for injected tumors in the control treatment group. FIG. 12B provides growth curves for injected tumors in the triple agent treated group (top panel), contralateral tumors in the triple agent treated group (middle panel), and contralateral tumors in the control treatment group (bottom panel).

FIG. 13. Graphs comparing mean tumor growth of injected (upper graph/panel) and non-injected (lower graph/panel) tumors using various different nanoparticle formulations. In each graph tumor size as surface area in mm² is represented on the Y axis and time in days is indicated on the X axis. In all nanoparticle formulations, MPL molecules are packaged inside the nanoparticle sphere while anti-CD40 mAbs coat the nanoparticle surface. The same concentrations of the active agents (anti-CD40 mAb and MPL) were used in the non-formulated and nanoparticle-formulated groups. Nanoparticles comprising chitosan, chitosan with protamine, albumin, mannose, PLGA, and protamine were tested—as indicated in the key to the right of each graph.

FIG. 14. Data obtained using chitosan nanoparticles. The 8 upper graphs provide tumor growth curves for individual tumors treated as indicated above each graph. The two lower graphs provide averaged tumor growth curves for tumors treated as shown in the key. In each graph tumor size as surface area in mm² is represented on the Y axis and time in days is indicated on the X axis. In all nanoparticle formulations MPL molecules are packaged inside the nanoparticles and anti-CD40 mAbs coated on the nanoparticle surface. As compared to the control group in which animals received systemic anti-PD-1 and intratumoral MPL and anti-CD40 (“non-formulated”), animals that received systemic anti-PD-1 and MPL with anti-CD40 co delivered as a nanoparticle (with MPL as the cargo and anti-CD40 on the surface of the nanoparticle) demonstrated improved control of injected and non-injected tumors.

FIG. 15. Data obtained using chitosan plus protamine nanoparticles. The 8 upper graphs provide tumor growth curves for individual tumors treated as indicated above each graph.

The two lower graphs provide averaged tumor growth curves for tumors treated as shown in the key. In each graph tumor size in mm² is represented on the Y axis and time in days is indicated on the X axis. In all nanoparticle formulations MPL molecules are packaged inside the nanoparticles and anti-CD40 mAbs coated on the nanoparticle surface. As compared to the control group in which animals received systemic anti-PD-1 and intratumoral MPL and anti-CD40 (“non-formulated”), animals that received systemic anti-PD-1 and MPL with anti-CD40 co delivered as a nanoparticle (with MPL as the cargo and anti-CD40 on the surface of the nanoparticle) demonstrated improved control of injected and non-injected tumors.

FIG. 16. Data obtained using albumin nanoparticles. The 8 upper graphs provide tumor growth curves for individual tumors treated as indicated above each graph. The two lower graphs provide averaged tumor growth curves for tumors treated as shown in the key. In each graph tumor size in mm² is represented on the Y axis and time in days is indicated on the X axis. In all nanoparticle formulations MPL molecules are packaged inside the nanoparticles and anti-CD40 mAbs coated on the nanoparticle surface. As compared to the control group in which animals received systemic anti-PD-1 and intratumoral MPL and anti-CD40 (“non-formulated”), animals that received systemic anti-PD-1 and MPL with anti-CD40 co delivered as a nanoparticle (with MPL as the cargo and anti-CD40 on the surface of the nanoparticle) demonstrated improved control of non-injected tumors.

FIG. 17. Data obtained using mannose nanoparticles. The 8 upper graphs provide tumor growth curves for individual tumors treated as indicated above each graph. The two lower graphs provide averaged tumor growth curves for tumors treated as shown in the key. In each graph tumor size in mm² is represented on the Y axis and time in days is indicated on the X axis. In all nanoparticle formulations MPL molecules are packaged inside the nanoparticles and anti-CD40 mAbs coated on the nanoparticle surface. As compared to the control group in which animals received systemic anti-PD-1 and intratumoral MPL and anti-CD40 (“non-formulated”), animals that received systemic anti-PD-1 and MPL with anti-CD40 co delivered as a nanoparticle (with MPL as the cargo and anti-CD40 on the surface of the nanoparticle) demonstrated improved control of injected and non-injected tumors.

FIG. 18. Data obtained using PLGA nanoparticles. The 8 upper graphs provide tumor growth curves for individual tumors treated as indicated above each graph. The two lower graphs provide averaged tumor growth curves for tumors treated as shown in the key. In each graph tumor size in mm² is represented on the Y axis and time in days is indicated on the X axis. In all nanoparticle formulations MPL molecules are packaged inside the nanoparticles and anti-CD40 mAbs coated on the nanoparticle surface. As compared to the control group in which animals received systemic anti-PD-1 and intratumoral MPL and anti-CD40 (“non-formulated”), animals that received systemic anti-PD-1 and MPL with anti-CD40 co delivered as a nanoparticle (with MPL as the cargo and anti-CD40 on the surface of the nanoparticle) demonstrated improved control of injected and non-injected tumors.

FIG. 19. Data obtained using fucoidan nanoparticles. The 8 upper graphs provide tumor growth curves for individual tumors treated as indicated above each graph. The two lower graphs provide averaged tumor growth curves for tumors treated as shown in the key. In each graph tumor size in mm² is represented on the Y axis and time in days is indicated on the X axis. In all nanoparticle formulations MPL molecules are packaged inside the nanoparticles and anti-CD40 mAbs coated on the nanoparticle surface. As compared to the control group in which animals received systemic anti-PD-1 and intratumoral MPL and anti-CD40 (“non-formulated”), animals that received systemic anti-PD-1 and MPL with anti-CD40 co-delivered as a nanoparticle (with MPL as the cargo and anti-CD40 on the surface of the nanoparticle) demonstrated improved control of injected and non-injected tumors.

FIG. 20A-D. Photographs of mice demonstrating evidence of systemic tumor-specific adaptive immune response. The two left-hand panels (FIG. 20A and FIG. 20C) provide photographs of mice treated with intratumoral MPL (5μg), the CD40 agonist mAb FGK45 (20μg), and intraperitoneal anti-PD-1 mAb RMP1-14 (250 μg). The two right-hand panels (FIG. 20B and FIG. 20D) provide photographs of mice treated with intratumoral MPL (5μg), FGK45 (20μg), and anti-IL10R mAb 1B1.3A (100μg) without systemic PD-1 blockade. In both of the two upper panels (FIG. 20A and FIG. 20B) patches of fur depigmentation (white) are evident at the site of intratumoral treatment. At 90 days, in the absence of ongoing treatment, the animals were re-implanted with tumor cells in the contralateral flank. As shown in the two lower panels (FIG. 20C and FIG. 20D) the animals resisted new tumor formation, and formed small patches of depigmented fur at the re-challenged site. The notation 440P refers to the treatment with MPL (IT), anti-CD40 (IT), and anti-PD1 (IP). The notation 41040 refers to treatment with MPL (IT), anti-IL10R (IT), and anti-CD40 (IT).

FIG. 21A-F provides additional data from the experiments described in FIG. 20. The data provided in FIGS. 21A-C demonstrates that treatment with IT CD40 mAb, IT MPL and systemic PD-1 mAb causes rapid neutrophil accumulation and activation as depicted by upregulation of CD86; and rapid DC activation as depicted by upregulation of CD86, at the injected tumor. The data provided in FIGS. 21D-F demonstrates that treatment with IT CD40 mAb, IT MPL and systemic PD-1 mAb causes subsequent infiltration and proliferation of CD8 T cells at the contralateral tumor. FIG. 21A is a graph with data showing CD86 levels within injected tumors. The X axis shows time points after initial treatment ranging from 3 hours to 1 week. The Y axis shows CD86 mean fluorescence intensity (MFI). FIG. 21B includes two graphs. In the left-hand graph the X axis shows time points after initial treatment and the Y axis shows axis shows % of CD11b, Ly6g double positive cells among live CD45 positive cells. In the right-hand graph the X axis shows time points after initial treatment and the Y axis shows % of CD86 positive cells among CD11b Ly6g double positive cells. FIG. 21C shows haematoxylin and eosin (H&E) staining of injected tumors at baseline (0 h), 24 hrs, and 72 hours after initial treatment with MPL (IT), anti-CD40 (IT), and anti-PD1 (IP). Heavy neutrophil infiltration is seen at 24 hours. Nearly complete eradication of tumor is seen by 72 hours. FIG. 21D shows H&E staining of a contralateral (non-injected) tumor at one week after treatment (MPL (IT), anti-CD40 (IT), and anti-PD1 (IP)) or control (vehicle and isotype mAb). This shows show significant lymphocyte infiltration in the treatment, but not the control, group. FIG. 21E provides a graph on which the Y axis represents the % of CD8 cells among live CD45 cells within the contralateral tumor, and the X axis represents time points from 3 hours to 2 weeks. The “treatment” was MPL (IT), anti-CD40 (IT), and anti-PD1 (IP). FIG. 21F provides a graph on which the Y axis represents the % of Ki67 positive cells among CD8 positive cells in the contralateral tumor, and the X axis represents time points from 3 hours to 2 weeks. The “treatment” was MPL (IT), anti-CD40 (IT), and anti-PD1 (IP).

FIG. 22 H&E staining showing that pigmented dendritic melanophages accumulate in the T-cell rich splenic peri-arterial lymphatic sheath 24 hours after single treatment with intratumoral MPL (5μg), FGK45 (20μg), and anti-IL10R mAb 1B1.3A (100μg).

FIG. 23A-C provide data from experiments performed using the bilateral tumor model referred to above, now using an ovarian cancer cell line to form tumors. C57BL/6 animals were challenged bilaterally with ovarian carcinoma ID8 syngeneic tumor cells. Established tumors were treated with intratumoral MPL (5μg), FGK45 (20μg), and anti-IL10R mAb 1B1.3A (100μg) and intraperitoneal anti-PD-1 mAb RMP1-14 (250 μg). Control animals received either intraperitoneal RMP1-14 alone or isotype mAb in vehicle. The notation 41040P refers to treatment with MPL (IT), anti-IL10R (IT), anti-CD40 (IT), and anti-PD1 (IP) FIG. 23A shows 6 graphs each showing individual tumor growth curves, with tumor surface area plotted in mm² plotted on the Y axes and time in days plotted on the X axes. The labels at the top of each of the 6 graphs summarize the treatment used (i.e. isotype control, anti-PD-1 mAb RMP1-14 alone, or the combination treatment described above). The upper 3 graphs are growth curves from the injected tumors and the lower 3 graphs are growth curves from the non-injected tumor. FIG. 23B and FIG. 23C provide averaged tumor growth curves with tumor surface area plotted in mm² plotted on the Y axes and time in days plotted on the X axes. On each graph data from the two controls (isotype/vehicle control and RMP1-14/antiPD-1alone comtrol) and the combination treatment (41040P—i.e. treatment with MPL (IT), anti-IL10R (IT), anti-CD40 (IT), and anti-PD1 (IP)) are shown. FIG. 23B provides data from the injected tumors. FIG. 23C provides data from the non-injected tumors.

FIG. 24A-B shows data obtained from an experiment that was the same as that described above (for which the data is provided in FIG. 23) with the exception that syngeneic sarcoma LiHA tumor cells/tumors were used in place of syngeneic ovarian carcinoma ID8 tumor cells/tumors. FIG. 24A shows 6 graphs each showing individual tumor growth curves, with tumor surface area plotted in mm² plotted on the Y axes and time in days plotted on the X axes. The left-hand graph panels are from isotype controls, the middle graph panels are from the IP anti-PD-1 mAb RMP1-14 alone controls, and the right-hand panels are from the “41040P” combination treatment described above). The upper 3 graphs are growth curves from the injected tumors and the lower 3 graphs are growth curves from the non-injected tumors. FIG. 24B provides two graphs with averaged tumor growth curves. Tumor surface area in mm² is plotted on the Y axes and time in days is plotted on the X axes. On each graph data from the two controls (isotype/vehicle control and RMP1-14/antiPD-1alone control) and the combination treatment (41040P—i.e. treatment with MPL (IT), anti-IL10R (IT), anti-CD40 (IT), and anti-PD1 (IP)) are shown. The left-hand panel of FIG. 24B provides data from the injected tumors. The right-hand panel of FIG. 24B provides data from the non-injected tumors.

FIG. 25A-C provides results of experiments in which C57BL/6 animals were challenged intravenously (IV) with syngeneic HKP (krasG12D/+, p53f/f) lung carcinoma cells. Once bilateral lung tumors were established animals were treated once weekly for four weeks, and luminescence was assayed to monitor tumor growth. Animals received either isotype control mAbs, non-formulated mixtures of intratumoral MPL (5 μg) and FGK45 (20 μg) together with IP 250 μg of RMP1-14, or intravenous MPL (5 μg) and FGK45 (20 μg) formulated as a chitosan nanoparticle as described above together with 250 μg of IP RMP1-14. FIG. 25A provides individual tumor growth curves as quantified by relative luminescence (Y axes) over time in days (X axes) for the indicated treatment groups. FIG. 25B provides averaged data for each treatment group with normalized relative luminescence (Y axis) plotted against time in days (X axis). FIG. 25C provides the corresponding Kaplan-Meier survival curves for each treatment group—as indicated.

FIGS. 26A-B provide data showing that the nanoparticle formulations described in the present patent application can be improved by adding either an anti-IL10R (1B1.3A) mAb to the surface or polyIC as cargo. The graphs depict tumor growth of B16 tumors with tumor surface area in mm² plotted on the Y axes and time after tumor implantation in days plotted on the X axes. FIG. 26A provides data obtained using chitosan nanoparticles with either MPL inside (as cargo) and both CD40 agonist mAb and IL10R blocking mAb on the surface (data represented by triangles) or with MPL inside (as cargo) and only CD40 agonist mAb on the surface (data represented by squares). The amounts of the active agents administered were as follows: 20 μg CD40 mAb, 5 μg MPL, and 100 μg IL10R mAb (1B1.3A). Both groups (with or without IL10R mAb) were also treated with intraperitoneal anti-PD-1 (250 μg). The upper graph in FIG. 26A shows data from the injected tumor. The lower graph in FIG. 26A shows data from the non-injected tumor. FIG. 26B provides data obtained using chitosan nanoparticles with either MPL alone inside (as cargo) and CD40 agonist mAb on the surface (data represented by squares) or with MPL plus polyIC inside (as cargo) and CD40 agonist mAb on the surface (data represented by diamonds). The amounts of the active agents administered were as follows: 20 μg CD40 mAb, 5μg MPL. Both groups (with or without polyIC) were also treated with intraperitoneal anti-PD-1 (250 μg). The upper graph in FIG. 26B shows data from the injected tumor. The lower graph in FIG. 26B shows data from the non-injected tumor.

FIG. 27A-B. FIG. 27A—average tumor growth curves for injected tumors. FIG. 27B—individual tumor growth curves for injected tumors. In both FIG. 27A and FIG. 27B tumor surface area in mm² (Y axis) is plotted against time after tumor implantation in days (X axis). Treatments in each graph were with the agents indicated in the figures (i.e. isotype control, anti-PD-1 mAb monotherapy, anti-CD40 mAb monotherapy, MPL monotherapy, or combination therapy with MPL plus anti-CD40 mAb plus anti-PD-1 mAb (referred to as 440P in FIG. 27A). Additional details including doses are provided in the Examples.

FIG. 28A-B. FIG. 28A—average tumor growth curves for injected tumors. FIG. 28B—individual tumor growth curves for injected tumors. In both FIG. 28A and FIG. 28B tumor surface area in mm² (Y axis) is plotted against time after tumor implantation in days (X axis). Treatments in each graph were with the agents indicated in the figures (i.e. isotype control, anti-CD40 mAb plus anti-PD-1 mAb, or MPL plus anti-CD40 mAb plus anti-PD-1 mAb (referred to as 440P in FIG. 28A). Additional details including doses are provided in the Examples.

FIG. 29A-B. FIG. 29A—average tumor growth curves for injected tumors. FIG. 29B—individual tumor growth curves for injected tumors. In both FIG. 29A and FIG. 29B tumor surface area in mm² (Y axis) is plotted against time after tumor implantation in days (X axis). Treatments in each graph were with the agents indicated in the figures (i.e. isotype control, MPL plus anti-PD-1 mAb, or MPL plus anti-CD40 mAb plus anti-PD-1 mAb (referred to as 440P in FIG. 29A). Additional details including doses are provided in the Examples.

FIG. 30A-B. FIG. 30A—average tumor growth curves for injected tumors. FIG. 30B—individual tumor growth curves for injected tumors. In both FIG. 30A and FIG. 30B tumor surface area in mm² (Y axis) is plotted against time after tumor implantation in days (X axis). Treatments in each graph were with the agents indicated in the figures (i.e. isotype control, MPL plus anti-CD40 mAb, or MPL plus anti-CD40 mAb plus anti-PD-1 mAb (referred to as 440P in FIG. 30A). In these experiments anti-PD-1 mAb was administered intraperitoneally (IP) and all other agents were administered intratumorally (IT). Additional details including doses are provided in the Examples.

FIG. 31A-B. FIG. 31A—average tumor growth curves for non-injected tumors. FIG. 31B—individual tumor growth curves for non-injected tumors. In both FIG. 31A and FIG. 31B tumor surface area in mm² (Y axis) is plotted against time after tumor implantation in days (X axis). Treatments in each graph were with the agents indicated in the figures (i.e. isotype control, anti-PD-1 mAb monotherapy, anti-CD40 mAb monotherapy, MPL monotherapy, or combination therapy with MPL plus anti-CD40 mAb plus anti-PD-1 mAb (referred to as 440P in FIG. 31A). Additional details including doses are provided in the Examples.

FIG. 32A-B. FIG. 32A—average tumor growth curves for non-injected tumors. FIG. 32B—individual tumor growth curves for non-injected tumors. In both FIG. 32A and FIG. 32B tumor surface area in mm² (Y axis) is plotted against time after tumor implantation in days (X axis). Treatments in each graph were with the agents indicated in the figures (i.e. isotype control, anti-CD40 mAb plus anti-PD-1 mAb, or MPL plus anti-CD40 mAb plus anti-PD-1 mAb (referred to as 440P in FIG. 32A). Additional details including doses are provided in the Examples.

FIG. 33A-B. FIG. 33A—average tumor growth curves for non-injected tumors. FIG. 33B—individual tumor growth curves for non-injected tumors. In both FIG. 33A and FIG. 33B tumor surface area in mm² (Y axis) is plotted against time after tumor implantation in days (X axis). Treatments in each graph were with the agents indicated in the figures (i.e. isotype control, MPL plus anti-PD-1 mAb, or MPL plus anti-CD40 mAb plus anti-PD-1 mAb (referred to as 440P in FIG. 33A). Additional details including doses are provided in the Examples.

FIG. 34A-B. FIG. 34A—average tumor growth curves for non-injected tumors. FIG. 34B—individual tumor growth curves for non-injected tumors. In both FIG. 34A and FIG. 34B tumor surface area in mm² (Y axis) is plotted against time after tumor implantation in days (X axis). Treatments in each graph were with the agents indicated in the figures (i.e. isotype control, MPL plus anti-CD40 mAb, or MPL plus anti-CD40 mAb plus anti-PD-1 mAb (referred to as 440P in FIG. 34A). Additional details including doses are provided in the Examples.

FIG. 35A-B. FIG. 35A graphs showing that anti-CD40, anti-IL10R, or the combination of both, are also effective when administered systemically instead of intratumorally. FIG. 35B graphs showing that PolyIC (a TLR3 agonist) can be substituted for MPL, albeit possibly with slightly reduced activity. Both FIG. 35A and FIG. 35B contain individual tumor growth curves having tumor surface area in mm² (Y axis) plotted against time after tumor implantation in days (X axis). Measurements are either for the injected or non-injected contralateral tumor—as indicated. Treatments in each graph were with the agents indicated in the figures. Additional details including doses are provided in the Examples.

FIG. 36A-B. Data showing that concurrent addition of systemic chemotherapy (in this case oxaliplatin or “OXA”) increases survival associated with intratumoral MPL, anti-IL10R, and anti-CD40 in the bilateral tumor model described herein. FIG. 36A contains individual tumor growth curves having tumor surface area in mm² (Y axis) plotted against time after tumor implantation in days (X axis). Measurements are either for the injected (top row of graphs) or non-injected contralateral (bottom row of graphs) tumors—as indicated. Treatments in each graph were with the agents indicated in the figures. The data in the two left-hand graphs was obtained from isotype/vehicle control treated mice. The data in the two middle graphs was obtained from mice treated with MPL (IT), anti-IL10R (IT), and anti-CD40 (IT) (this combination treatment is referred to as “41040” in the Figure). The data in the two right-hand graphs was obtained from mice treated with the “41040” combination as well as IP (systemic) oxaliplatin (OXA). Additional details including doses are provided in the Examples. FIG. 36B provides survival curves for the indicated treatments, and demonstrates that concurrent addition of systemic oxaliplatin increased the survival advantage associated with intratumoral MPL, anti-IL10R, and anti-CD40.

FIG. 37A-C. Individual tumor growth curves from tumor model experiments in which the mice initially had one tumor (tumor cells injected on one flank), and then after treatment of that tumor, at day 90, a second tumor was implanted on the other flank. Tumor surface area in mm² (Y axis) is plotted against time after tumor implantation in days (X axis). A regimen of intratumoral MPL, anti-CD40, and anti-IL10R eradicated injected tumors (FIG. 37A). At day 90, 10/10 treated mice resisted tumor re-challenge (FIG. 37B), compared to 0/10 naïve controls (FIG. 37C). The doses of the active agents used in these experiments were halved as compared to the doses used in the other experiments described in the Examples section of this patent application. These data indicate the formation and persistence and anti-tumor response, suggesting robust anti-tumor immunologic memory.

FIG. 38A-B. Individual tumor growth curves from tumor model experiments—using the tumor model described for FIG. 37. Tumor surface area in mm² (Y axis) is plotted against time after tumor implantation in days (X axis). At day 90, 8/8 mice treated with systemic (IP) anti-PD-1 together with intratumoral MPL and anti-CD40 resisted tumor re-challenge (FIG. 38B), compared to 0/10 naïve controls (FIG. 38A), indicating that anti-tumor immunologic memory is established and persists with this regimen as well.

FIG. 39. Experiments were performed to determine if the treatment regimens described herein could also be useful in the context of treatment with the immune checkpoint inhibitor anti-CTLA-4. FIG. 39 provides individual tumor growth curves from bilateral tumor model experiments performed as described for other figures. Tumor surface area in mm² (Y axis) is plotted against time after tumor implantation in days (X axis). The two left-hand graphs provide data obtained from control (isotype/vehicle) treated mice. The two right-hand graphs provide data from mice treated with IP anti-CTLA-4, IT MPL, and IT anti-CD40. The two upper graphs provide data obtained from the injected tumor. The two lower graphs provide data obtained from the non-injected contralateral tumor. The data shows that a regimen of IT anti-CD40 mAb and MPL together with the immune checkpoint inhibitor anti-CTLA-4 (administered systemically via intraperitoneal injection) confers antitumor activity.

### DETAILED DESCRIPTION

While some of the main embodiments of the present invention are described in the above Summary of the Invention section of this patent application, as well as in the section of this application, this Detailed Description section provides certain additional description relating to the compositions and methods of the present invention, and is intended to be read in conjunction with all other sections of the present patent application.

Definitions and Abbreviations

As used herein the abbreviation “APC” refers to an Antigen Presenting Cell.

As used herein the abbreviation “CD40” refers to a cluster of differentiation 40—a receptor that may be found on APCs, where it is involved in stimulating APC activation.

As used herein the abbreviation “DC” refers to a Dendritic Cell

As used herein the abbreviation “IL10” refers to interleukin 10.

As used herein the abbreviation “IL10R” refers to an IL10 receptor, such as an IL10R present on APCs. The term “IL10R” include any and all subunits of the IL10 receptor, including, but not limited to, IL10RA, IL10RB, IL10R1, and IL10R2.

As used herein the abbreviation “IP” refers to intraperitoneal.

As used herein the abbreviation “IT: refers to intratumoral. For example a drug injected directly into a tumor is delivered intratumorally.

As used herein the abbreviation “IV” refers to intravenous. It is common to administer agents to mice via an IP route, which is considered to be analogous to administering an agent to a human subject by a IV route.

As used herein the abbreviation “MPL” refers to monophosphoryl lipid A. MPL is a TLR4 agonist.

As used herein the abbreviation PD-1” refers to Programmed Death 1, which is also known as Programmed Death Protein 1 or Programmed Cell Death Protein 1.

As used herein the abbreviation PD-L1 refers to a ligand for PD-1.

As used herein the abbreviation “TLR” refers to Toll-like receptor(s). TLRs on APCs are involved in stimulating APC activation.

As used herein the terms “inhibiting” and “blocking” are used interchangeably, as are the terms “inhibit” or “block” and the terms “inhibitor” or “blocker.”

As used herein, the terms “about” and “approximately,” when used in relation to numerical values, mean within + or −20% of the stated value. Other terms are defined elsewhere in this patent specification, or else are used in accordance with their usual meaning in the art.

Other abbreviations and definitions may be provided elsewhere in this patent specification, or may be well known in the art.

Active Agents for Use in the Compositions and Methods of the Invention

As described in the Summary of the Invention and other sections of this patent application, the methods and compositions provided by present invention involve various different active agents, including, but not limited to, CD40 agonist s (e.g. CD40 agonist antibodies), TLR agonists, immune checkpoint inhibitors (such as immune checkpoint inhibitor antibodies, PD-1 inhibitors (such as PD-1 inhibitor antibodies), PD-L1 inhibitors (such as PD-L1 inhibitor antibodies), CTLA-4 inhibitors (such as CTLA-4 inhibitor antibodies), and IL10 receptor blocking antibodies. Each of the embodiments described herein that involves one or more of such active agents, such as those known in the art (including, but not limited to the specific exemplary agents described herein), can, in some embodiments, be carried out using any suitable analogues, homologues, variants, or derivatives of such agents. Such analogues, homologues, variants, or derivatives should retain the key functional properties of the specific molecules described herein. For example, in the case of the CD40 agonist antibodies, any suitable analogue, homologue, variant, or derivative of such an antibody can be used provided that it retains CD40 agonist activity. In the case of the TLR agonists, any suitable analogue, homologue, variant, or derivative of such an agent can be used provided that it retains TLR agonist activity. In the case of PD-1 inhibitors, any suitable analogue, homologue, variant, or derivative of such an agent can be used provided that it retains PD-1 inhibitory activity. In the case of PD-L1 inhibitors, any suitable analogue, homologue, variant, or derivative of such an agent can be used provided that it retains PD-L1 inhibitory activity. In the case of CTLA-4 inhibitors, any suitable analogue, homologue, variant, or derivative of such an agent can be used provided that it retains CTLA-4 inhibitory activity.

Similarly, in the case of IL10 receptor blocking antibodies, any suitable analogue, homologue, variant, or derivative of such an agent can be used provided that it retains IL10 receptor blocking activity.

Several embodiments of the present invention involve antibodies. As used herein, the term “antibody” encompasses intact polyclonal antibodies, intact monoclonal antibodies, single-domain antibody, nanobody, antibody fragments (such as Fab, Fab′, F(ab′)2, and Fv fragments), single chain Fv (scFv) mutants, multispecific antibodies such as bispecific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked, or conjugated to other molecules such as toxins, radioisotopes, or any of the other specific molecules recited herein.

The term “humanized antibody” refers to an antibody derived from a non-human (e.g., murine) immunoglobulin, which has been engineered to contain minimal non-human (e.g., murine) sequences. Typically, humanized antibodies are human immunoglobulins in which residues from the complementary determining region (CDR) are replaced by residues from the CDR of a non-human species (e.g., mouse, rat, rabbit, or hamster) that have the desired specificity, affinity, and capability (Jones et al., 1986, Nature, 321:522-525; Riechmann et al., 1988, Nature, 332:323-327; Verhoeyen et al., 1988, Science, 239:1534-1536). In some instances, the Fv framework region (FW) residues of a human immunoglobulin are replaced with the corresponding residues in an antibody from a non-human species that has the desired specificity, affinity, and capability.

Humanized antibodies can be further modified by the substitution of additional residues either in the Fv framework region and/or within the replaced non-human residues to refine and optimize antibody specificity, affinity, and/or capability. In general, humanized antibodies will comprise substantially all of at least one, and typically two or three, variable domains containing all or substantially all of the CDR regions that correspond to the non-human immunoglobulin whereas all or substantially all of the FR regions are those of a human immunoglobulin consensus sequence. Humanized antibody can also comprise at least a portion of an immunoglobulin constant region or domain (Fc), typically that of a human immunoglobulin. Examples of methods used to generate humanized antibodies are described in U.S. Pat. Nos. 5,225,539 or 5,639,641.

The term “human antibody” means an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide such as, for example, an antibody comprising murine light chain and human heavy chain polypeptides.

The term “chimeric antibodies” refers to antibodies wherein the amino acid sequence of the immunoglobulin molecule is derived from two or more species. Typically, the variable region of both light and heavy chains corresponds to the variable region of antibodies derived from one species of mammals (e.g., mouse, rat, rabbit, etc.) with the desired specificity, affinity, and capability while the constant regions are homologous to the sequences in antibodies derived from another (usually human) to avoid eliciting an immune response in that species.

A “monoclonal antibody” (mAb) refers to a homogeneous antibody population involved in the highly specific recognition and binding of a single antigenic determinant, or epitope. This is in contrast to “polyclonal antibodies” that typically include different antibodies directed against different antigenic determinants. The term “monoclonal antibody” encompasses both intact and full-length monoclonal antibodies, as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal antibody” refers to such antibodies made in any number of ways including, but not limited to, by hybridoma, phage selection, recombinant expression, and transgenic animals.

In particular, monoclonal antibodies can be prepared using hybridoma methods, such as those described by Kohler and Milstein (1975) Nature 256:495. Using the hybridoma method, a mouse, hamster, or other appropriate host animal, is immunized as described above to elicit the production by lymphocytes of antibodies that will specifically bind to an immunizing antigen. Lymphocytes can also be immunized in vitro. Following immunization, the lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a chosen antigen as determined by immunoprecipitation, immunoblotting, or by an in vitro binding assay (e.g. radioimmunoassay (MA); enzyme-linked immunosorbent assay (ELISA)) can then be propagated either in in vitro culture using standard methods (Goding, Monoclonal Antibodies: Principles and Practice, Academic Press, 1986) or in vivo as ascites tumors in an animal. The monoclonal antibodies can then be purified from the culture medium or ascites fluid.

Alternatively, monoclonal antibodies can be made using recombinant DNA methods, as described in U.S. Pat. No. 4,816,567. The polynucleotides encoding a monoclonal antibody are isolated from mature B-cells or hybridoma cells, such as by RT-PCR using oligonucleotide primers that specifically amplify the genes encoding the heavy and light chains of the antibody, and their sequence is determined using conventional procedures. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, monoclonal antibodies are generated by the host cells. Also, recombinant monoclonal antibodies or antigen-binding fragments thereof of the desired species can be isolated from phage display libraries expressing CDRs of the desired species as described (McCafferty et al., 1990, Nature, 348:552-554; Clackson et al., 1991, Nature, 352:624-628; and Marks et al., 1991, J. Mol. Biol., 222:581-597).

Polyclonal antibodies can be produced by various procedures well known in the art. For example, a host animal such as a rabbit, mouse, rat, etc. can be immunized by injection with an antigen to induce the production of sera containing polyclonal antibodies specific for the antigen. The antigen can include a natural, synthesized, or expressed protein, or a derivative (e.g., fragment) thereof. Various adjuvants may be used to increase the immunological response, depending on the host species, and include, but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art. Antibodies can be purified from the host's serum.

Conjugated Agents for Use in the Compositions and Methods of the Invention

Several embodiments of the present invention involve an antibody-drug conjugate molecule comprising a CD40 agonist (e.g. a CD40 agonist antibody) and a TLR agonist, linked together via a linker moiety. Any suitable CD40 agonist and TLR agonist known in the art or described herein can be used. Similarly, any suitable linker moiety can be used to connect the CD40 agonist to the TLR agonist. Several such linkers are known in the art, such as those that are conventionally used in the production of antibody-drug conjugates. In some embodiments the linker is a lysine-bound linker, such as, for example, the “SMCC” linker that is commercially available from ImmunoGen. In some embodiments the linker is a cysteine-bound linker, such as, for example, the “vc-pABC” linker that is commercially available from Seattle Genetics.

Compositions.

In certain embodiments, the present invention provides compositions, such as pharmaceutical compositions. The term “pharmaceutical composition,” as used herein, refers to a composition comprising at least one active agent as described herein, and one or more other components useful in formulating a composition for delivery to a subject, such as diluents, buffers, carriers, stabilizers, dispersing agents, suspending agents, thickening agents, excipients, preservatives, and the like.

Some of the compositions, such as pharmaceutical compositions, described herein comprise two or more of the active agents described herein. In some of such embodiments the two or more agents may, optionally, be provided: adsorbed to the surface of alum, or within an emulsion, or within a liposome, or within a micelle, or within a polymeric scaffold, or adsorbed to the surface of, or encapsulated within, a polymeric particle, or within an immunostimulating complex or “iscom,” or within charge-switching synthetic adjuvant particle (cSAP), or within PLGA: poly(lactic-co-glycolic acid) particles, or within other nanoparticles suitable for pharmaceutical administration.

In those embodiments of the present invention that involve nanoparticles, any suitable nanoparticle chemistry or nanoparticle technology known in the art may be used. In some embodiments the nanoparticles may comprise one or more agents selected from the group consisting of mannose, chitosan, manosylated chitosan, protamine, chitosan with protamine, albumin, PLGA, and fucoidan. In some embodiments the nanoparticles may comprise a CD40 agonist (e.g. CD40 agonist antibody) on the surface of the nanoparticle. In some embodiments the nanoparticles may comprise an IL10 receptor-blocking antibody on the surface of the nanoparticle. In some embodiments the nanoparticles may comprise a TLR agonist within the nanoparticle. In some embodiments the nanoparticles may comprise an immune checkpoint inhibitor (such as a PD-1 inhibitor or PD-L1 inhibitor or CTLA-4 inhibitor) within the nanoparticle. In some embodiments the nanoparticles may comprise any combination of the above agents on the surface on or within the nanoparticles.

Methods of Treatment

In certain embodiments the present invention provides methods of treatment. As used herein, the terms “treat,” “treating,” and “treatment” encompass a variety of activities aimed at achieving a detectable improvement in one or more clinical indicators or symptoms associated with a tumor. For example, such terms include, but are not limited to, reducing the rate of growth of a tumor (or of tumor cells), halting the growth of a tumor (or of tumor cells), causing regression of a tumor (or of tumor cells), reducing the size of a tumor (for example as measured in terms of tumor volume or tumor mass), reducing the grade of a tumor, eliminating a tumor (or tumor cells), preventing, delaying, or slowing recurrence (rebound) of a tumor, improving symptoms associated with tumor, improving survival from a tumor, inhibiting or reducing spreading of a tumor (e.g. metastases), and the like.

The term “tumor” is used herein in accordance with its normal usage in the art and includes a variety of different tumor types. It is expected that the present methods and compositions can be used to treat any solid tumor. Suitable tumors that can be treated using the methods and compositions of the present invention include, but are not limited to, melanomas, lung tumors, colon tumors, prostate tumors, ovarian tumors, sarcomas, and breast tumors, and the various other tumor types mentioned in the present patent specification.

In carrying out the treatment methods described herein, any suitable method or route of administration can be used to deliver the active agents or combinations thereof described herein. In some embodiments systemic administration may be employed, for example, oral or intravenous administration, or any other suitable method or route of systemic administration known in the art. In some embodiments intratumoral delivery may be employed. For example, the active agents described herein may be administered directly into a tumor by local injection, infusion through a catheter placed into the tumor, delivery using an implantable drug delivery device inserted into a tumor, or any other means known in the art for direct delivery of an agent to a tumor.

As used herein the terms “effective amount” or “therapeutically effective amount” refer to an amount of an active agent as described herein that is sufficient to achieve, or contribute towards achieving, one or more desirable clinical outcomes, such as those described in the “treatment” description above. An appropriate “effective” amount in any individual case may be determined using standard techniques known in the art, such as dose escalation studies, and may be determined taking into account such factors as the desired route of administration (e.g. systemic vs. intratumoral), desired frequency of dosing, etc. Furthermore, an “effective amount” may be determined in the context of any co-administration method to be used. One of skill in the art can readily perform such dosing studies (whether using single agents or combinations of agents) to determine appropriate doses to use, for example using assays such as those described in the Examples section of this patent application—which involve administration of the agents described herein to subjects (such as animal subjects routinely used in the pharmaceutical sciences for performing dosing studies).

For example, in some embodiments the dose of an active agent of the invention may be calculated based on studies in humans or other mammals carried out to determine efficacy and/or effective amounts of the active agent. The dose amount and frequency or timing of administration may be determined by methods known in the art and may depend on factors such as pharmaceutical form of the active agent, route of administration, whether only one active agent is used or multiple active agents (for example, the dosage of a first active agent required may be lower when such agent is used in combination with a second active agent), and patient characteristics including age, body weight or the presence of any medical conditions affecting drug metabolism.

In those embodiments described herein that refer to specific doses of agents to be administered based on mouse studies, one of skill in the art can readily determine comparable doses for human studies based on the mouse doses, for example using the types of dosing studies and calculations described herein.

In some embodiments suitable doses of the various active agents described herein can be determined by performing dosing studies of the type that are standard in the art, such as dose escalation studies, for example using the dosages shown to be effective in mice in the Examples section of this patent application as a starting point. Interestingly, and as illustrated in the Examples, it has been found that the methods and compositions of the present invention are, effective using much lower doses of the active agents than would normally be used in other applications and contexts. In some embodiments, where the active agents used are antibodies, the agents are administered at a dose of from about 1 mg/kg to about 10 mg/kg, or at a dose of from about 0.1 mg/kg to about 10 mg/kg.

Dosing regimens can also be adjusted and optimized by performing studies of the type that are standard in the art, for example using the dosing regimens shown to be effective in mice in the Examples section of this patent application as a starting point. In some embodiments the active agents are administered daily, or twice per week, or weekly, or every two weeks, or monthly.

In certain embodiments the compositions and methods of treatment provided herein may be employed together with other compositions and treatment methods known to be useful for tumor therapy, including, but not limited to, surgical methods (e.g. for tumor resection), radiation therapy methods, treatment with chemotherapeutic agents, treatment with antiangiogenic agents, or treatment with tyrosine kinase inhibitors. Similarly, in certain embodiments the methods of treatment provided herein may be employed together with procedures used to monitor disease status/progression, such as biopsy methods and diagnostic methods (e.g. MRI methods or other imaging methods).

For example, in some embodiments the agents and compositions described herein may be administered to a subject prior to performing surgical resection of a tumor, for example in order to shrink a tumor prior to surgical resection. In other embodiments the agents and compositions described herein may be administered both before and after performing surgical resection of a tumor. In other embodiments the subject has no tumor recurrence after the surgical resection.

Subjects

As used herein the term “subject” encompasses all mammalian species, including, but not limited to, humans, non-human primates, dogs, cats, rodents (such as rats, mice and guinea pigs), cows, pigs, sheep, goats, horses, and the like—including all mammalian animal species used in animal husbandry, as well as animals kept as pets and in zoos, etc. In preferred embodiments the subjects are human. Such subjects will typically have (or previously had) a tumor (or tumors) in need of treatment. In some embodiments the subject has previously been treated with an immune checkpoint inhibitor (such as a PD-1 inhibitor, PD-L1 inhibitor, or a CTLA-4 inhibitor). In some embodiments the subject has not previously been treated with an immune checkpoint inhibitor (such as a PD-1 inhibitor, PD-L1 inhibitor, or a CTLA-4 inhibitor). In some embodiments the subject has a tumor that is insensitive to, or resistant to, treatment with an immune checkpoint inhibitor (such as a PD-1 inhibitor, PD-L1 inhibitor, or a CTLA-4 inhibitor), or that is suspected of being insensitive to, or resistant to, treatment with an immune checkpoint inhibitor (such as a PD-1 inhibitor, PD-L1 inhibitor, or a CTLA-4 inhibitor). In some embodiments the subject has a tumor that has recurred following a prior treatment with an immune checkpoint inhibitor (such as a PD-1 inhibitor, PD-L1 inhibitor, or a CTLA-4 inhibitor) and/or with one or more other tumor treatment methods, including, but not limited to, chemotherapy, radiation therapy, or surgical resection, or any combination thereof. In some embodiments the subject has a tumor that has not previously been treated, whether with an immune checkpoint inhibitor (such as a PD-1 inhibitor, PD-L1 inhibitor, or a CTLA-4 inhibitor) or with one or more other tumor treatment methods, including, but not limited to, chemotherapy, radiation therapy, or surgical resection, or any combination thereof.

### EXAMPLES

The invention is further described in the following non-limiting Examples, as well as the Figures referred to therein.

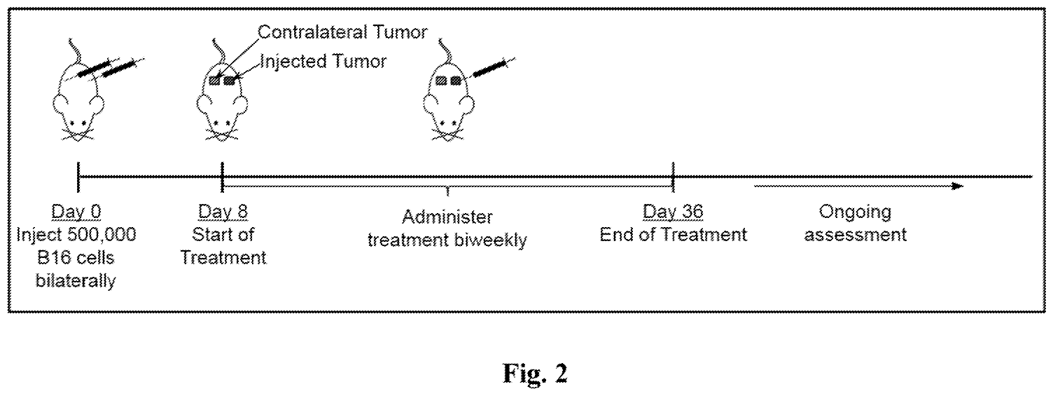

In each of these Examples, and unless stated otherwise, the indicated active agents were administered at the following doses: MPL—5 μg, anti-CD40—20 μg, anti-PD-1—250 μg, anti-IL10R—100 μg—regardless of the delivery route or formulation used. Also, unless indicated otherwise, controls were treated with isotype control antibodies and vehicle only. Furthermore, unless indicated otherwise, all experiments were performed using the mouse bilateral tumor model described below and summarized in FIG. 2.

### Example 1

### Treatment with Low Dose Anti-CD40 and MPL

Immune checkpoint blockade (for example using anti-CTLA-4, PD-1, and PD-L1 monoclonal antibodies (mAbs)) offers the potential for durable remissions for patients across a broad range of cancers, including, but not limited to, lung, breast, colon and prostate cancer. However, despite this broad applicability, the majority (well over 80%) of cancer patients are, or become, resistant to it. The studies presented in this Example demonstrate a novel approach to overcome resistance to immune checkpoint blockade in manner applicable to most cancers, regardless of type or stage.

Cancers refractory to immune checkpoint blockade generally fail to mount significant antitumor T lymphocyte responses. Many cancers, including breast and colon cancer demonstrate defective antigen presenting cell (APC) activation. Since APCs prime T lymphocytes, this can explain the absence of a productive anti-tumor T lymphocyte response in these cancers.

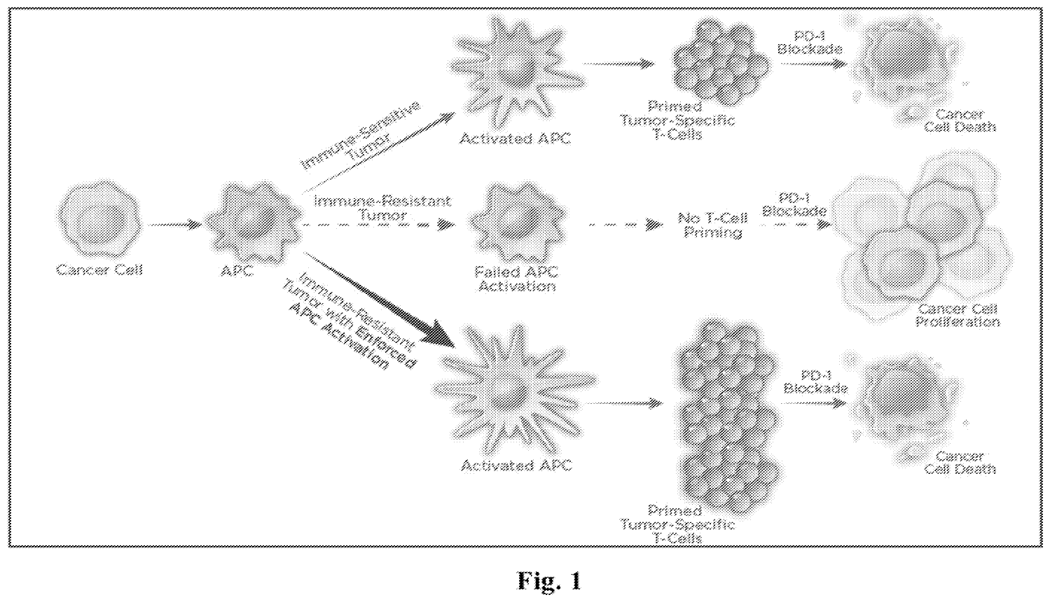

We hypothesized that enforced activation of tumor-associated APCs, by directly injecting tumors, could potentially convert an individual tumor and/or lymphoid organs into ‘cellular factories’ of primed anti-tumor T lymphocytes that could then, potentially, recognize and kill cancer cells throughout the body, and induce direct tumor cell killing by activating innate immune cells (including APCs) at the tumor site. FIG. 1 provides a schematic representation of this hypothesis.

A number of rationally-selected combinations of agents were chosen and tested in a murine model of aggressive melanoma, shown to be resistant to checkpoint blockade, with the aim of testing this hypothesis and identifying treatments with potent anti-tumor activity. The animals used had large tumors in two opposite flanks. One tumor was injected while the second remained non-injected (FIG. 2), allowing separate analysis of the effect at the injected tumor from the so-called ‘abscopal’ effect at the non-injected tumor, in order to understand how this treatment could benefit patients with metastatic cancer. However, in clinical applications multiple tumor sites can be injected. To test whether resistance to anti-PD-1 therapy can be reversed, we used the poorly immunogenic B16 murine melanoma model previously shown to be refractory to PD-1 blockade. C57BL/6 mice were initially implanted with 5×10⁵ syngeneic B16F10 cells intra-dermally in bilateral flanks. 8 days post tumor cell implantation, when bilateral tumors measured ˜0.5 cm, intratumoral (IT) treatment with various test agents was initiated together with intraperitoneal (IP) anti-PD-1 mAb. Treatment was administered twice weekly for 4 weeks into one of the bilateral tumors. The contralateral tumor remained un-injected for the duration of the experiment. FIG. 2 provides a schematic illustration of this experimental protocol.

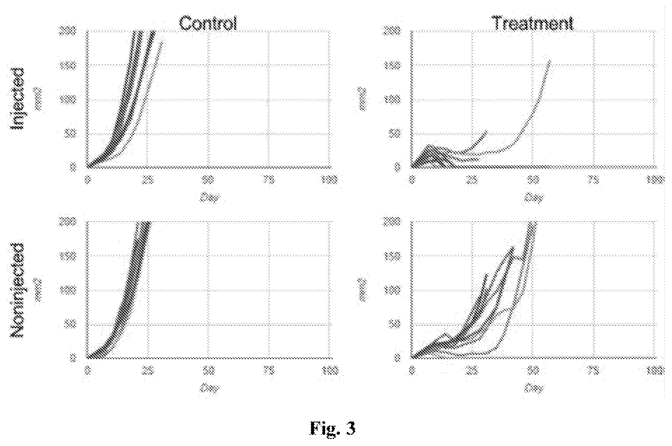

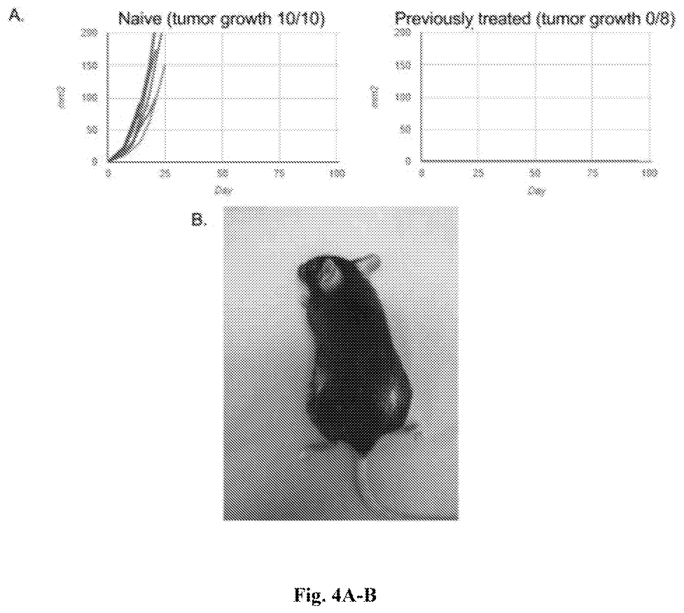

Mice were treated with MPL (intratumoral) at 5 μg, anti-CD40 (FGK45/FGK4.5, intratumoral) at 20 μg, and anti-PD-1 (RMP1-14, systemically via intraperitoneal injection) at 250 μg, while control mice were treated with isotype control antibodies and vehicle only. It was found that the combination of low dose anti-CD40 and MPL in the setting of systemic anti-PD-1: (A) yielded no discernible toxicity, (B) consistently eradicated injected tumors, (C) controlled or eradicated large non-injected tumors (FIG. 3), and (D) triggered long-lasting immunity in cured animals demonstrated by resistance to tumor re-implantation at 90 days without further treatment (FIG. 4).

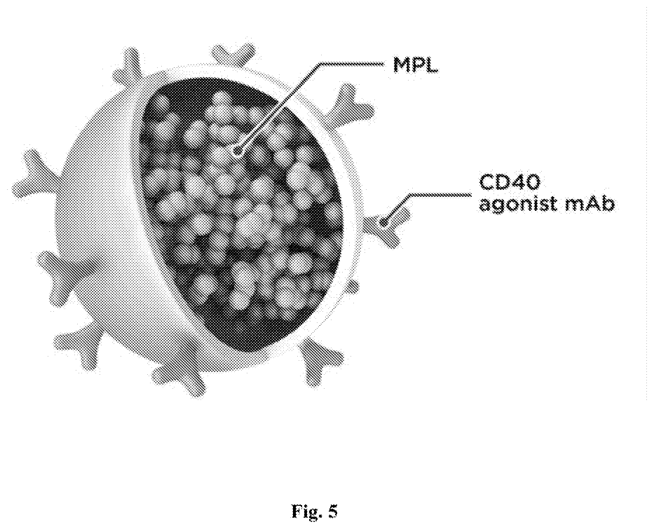

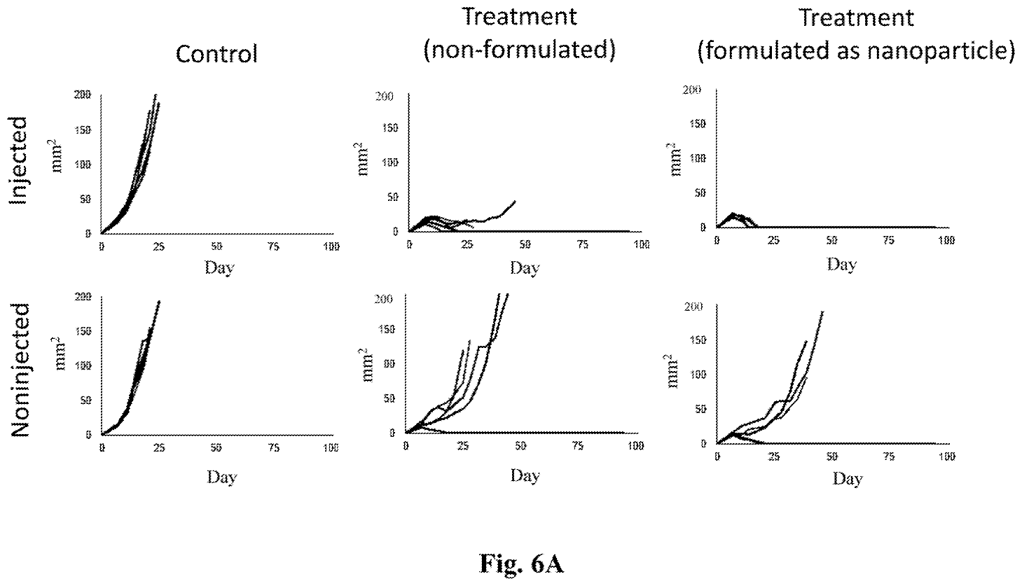

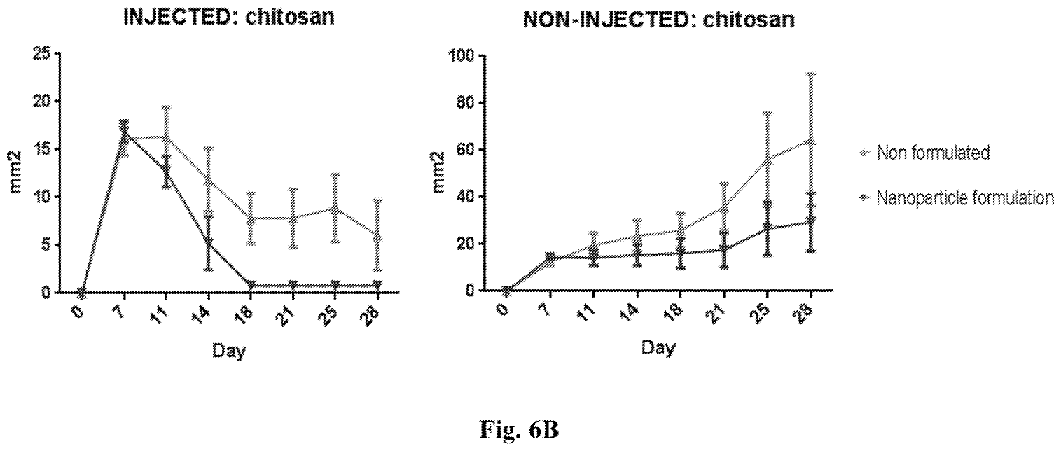

Studies were next performed to determine whether conjugation of the anti-CD40 mAb and MPL, for example using nanoparticle technology (FIG. 5), could further enhance treatment potency. Six such nanoparticles coated with anti-CD40 mAb and carrying MPL internally as cargo were constructed and tested in vivo. The six different nanoparticle compositions were those formulated with either chitosan, albumin, mannose, PLGA, fucoidan, or a combination of chitosan with protamine. The results of this testing indicated that multiple of such nanoparticle formulations had superior therapeutic efficacy as compared to non-formulated MPL and anti-CD40 administered at doses equivalent to those in the nanoparticle formulation (FIG. 6).

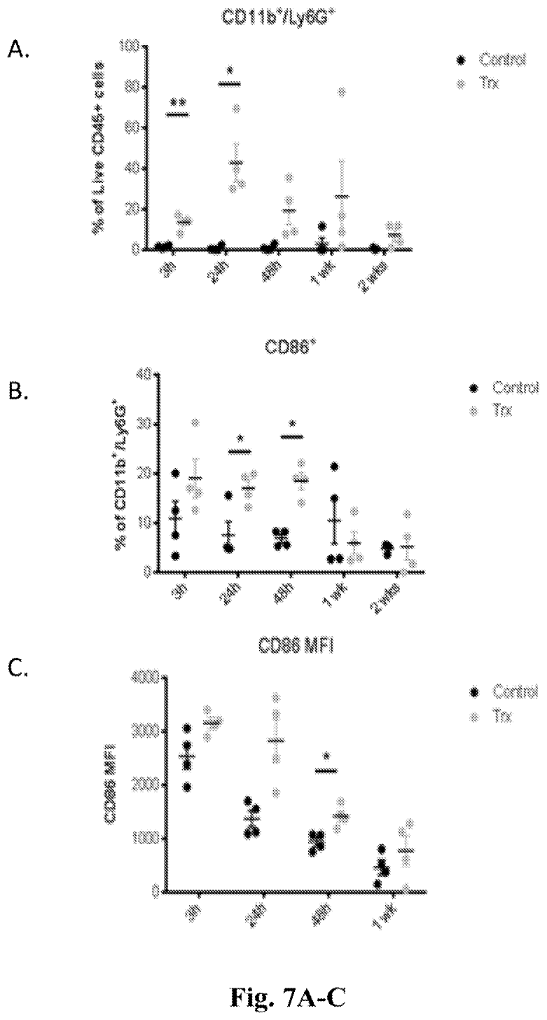

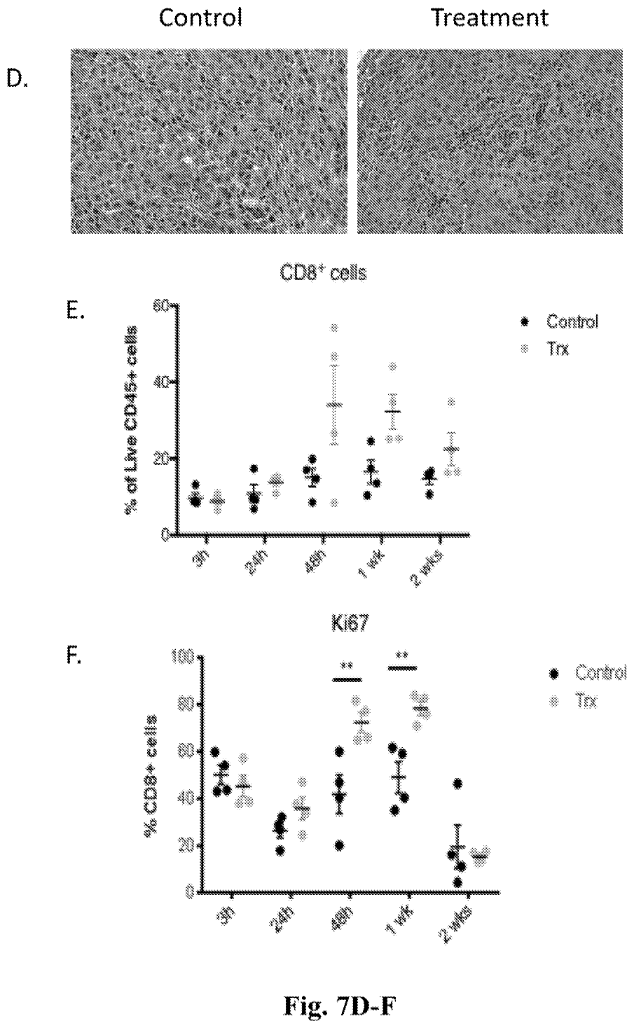

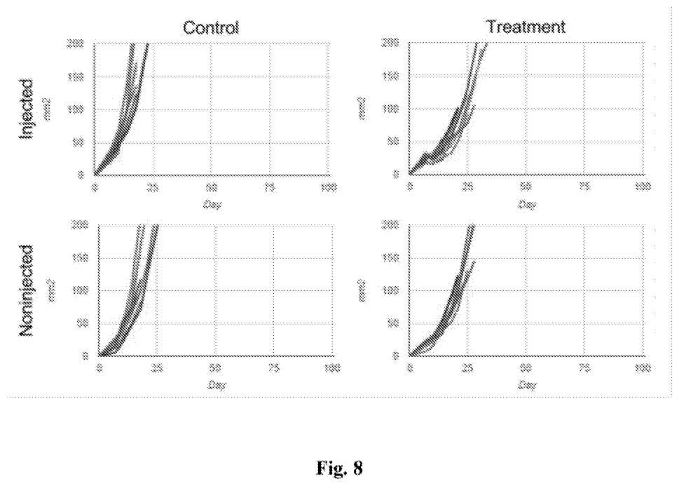

Furthermore, additional studies demonstrated successful conversion of a living tumor into a ‘cellular factory’—which produces lymphocytes that, in turn, infiltrate and regress distant non-injected tumors. In particular, it was found that injected tumors were rapidly infiltrated with neutrophils—an important class of APCs (FIG. 7A). Furthermore it was found that these neutrophils up-regulated the co-stimulatory molecule CD86 (FIG. 7B) which marks activated APCs (and specifically neutrophils, see Leliefeld, P. H. C., Koenderman, L. & Pillay, J. How Neutrophils Shape Adaptive Immune Responses. Front. Immunol. 6, 471 (2015)) and primes T lymphocytes against a specific target. Dendritic cells (efficient APCs 6 within the tumor) were also found to similarly upregulate CD86 in response to treatment (FIG. 7C). Thus multiple lines of evidence indicate that, using the compositions and methods described herein, the tumor rapidly becomes optimized for priming lymphocytes to recognize and kill tumor cells. After one week of treatment it was observed that such lymphocytes infiltrated contralateral tumors (FIGS. 7D, E, & F) as their growth was curtailed (FIG. 3). This data is consistent with our hypothesis of enforced APC activation converting living tumors into sources of lymphocytes that overcome resistance to PD-1 blockade. To confirm the role of lymphocytes in treatment activity the experiment described above—for which the results are shown in FIG. 3—was repeated in animals lacking functional lymphocytes (RAG-1 KO mice). Tumor control was severely diminished in the injected tumor, and virtually abolished in the non-injected tumor, confirming the critical role of lymphocytes. This data from RAG-1 KO animals—which is shown in FIG. 8.—was obtained in parallel with data from wild-type animals—which is shown in FIG. 3. Consistent with our hypothesis, non-injected tumors grew normally in mice lacking lymphocytes (FIG. 8).

Additional experiments were performed to determine whether the treatments outlined above might also be effective in other tumors and in other organs. Studies were performed using orthotopic lung cancer, sarcoma, and ovarian cancer models—as described in subsequent Examples. In each case potent treatment activity was observed.

Additional experiments were also performed to determine whether the agents could be effective systemically as well as intratumorally. Robust activity was also observed when the active agents were administered intravenously (IV) instead of intratumorally at the equivalent dose—as shown in other Examples.

Importantly, the compositions and methods described herein constitute an “off-the-shelf” method of priming and expanding tumor-specific T cells trained to recognize the patient's own tumor as it exists in the body and changes over time. This is in contrast to many other so-called “customized” approaches (e.g., vaccine, transgenic-T-cell, and CAR-T-cell therapy)—which instead often rely on directing lymphocytes to pre-defined targets associated with specific cancers. The treatment approaches described herein may therefore be less costly to produce, and more broadly applicable (for example across multiple cancer types and patients).

### Example 2

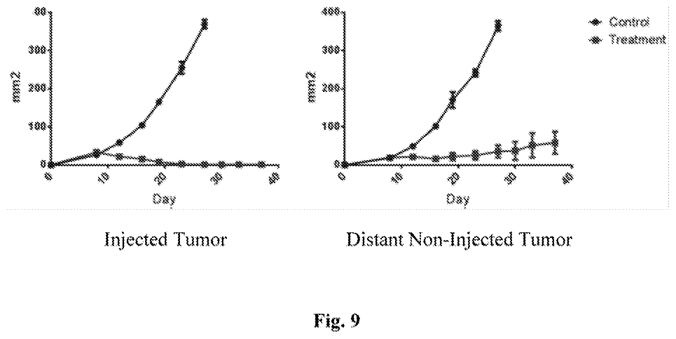

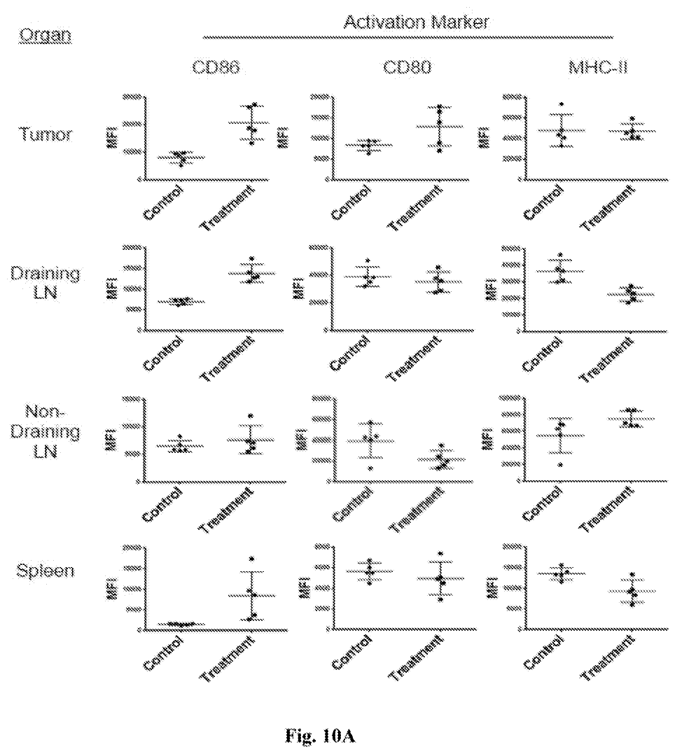

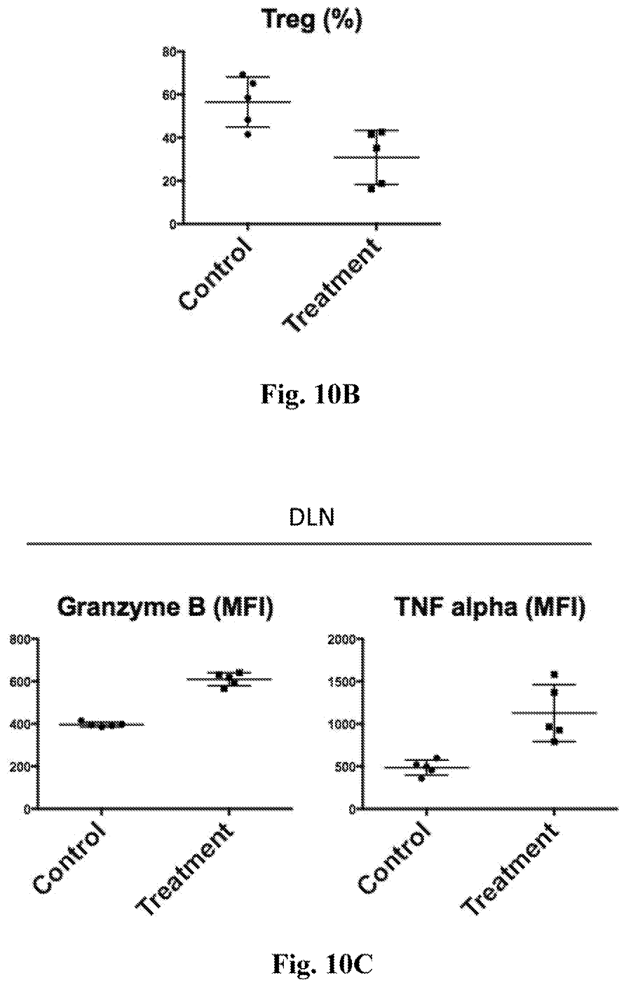

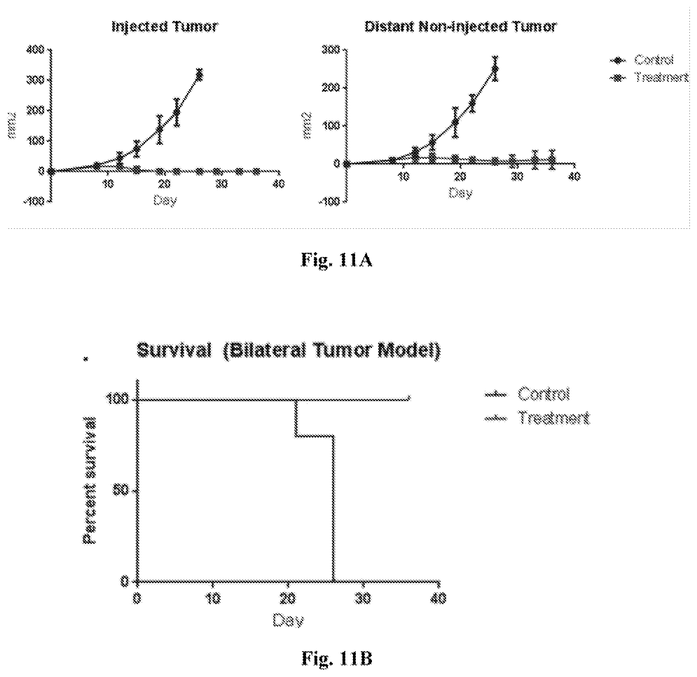

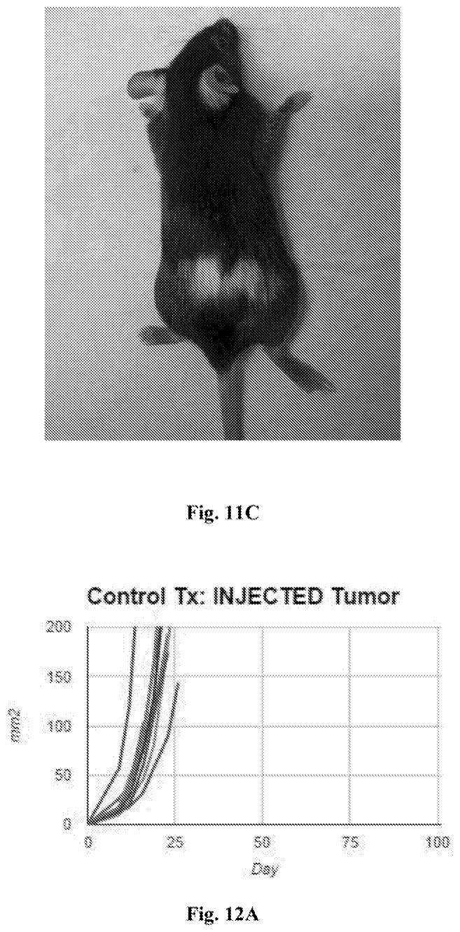

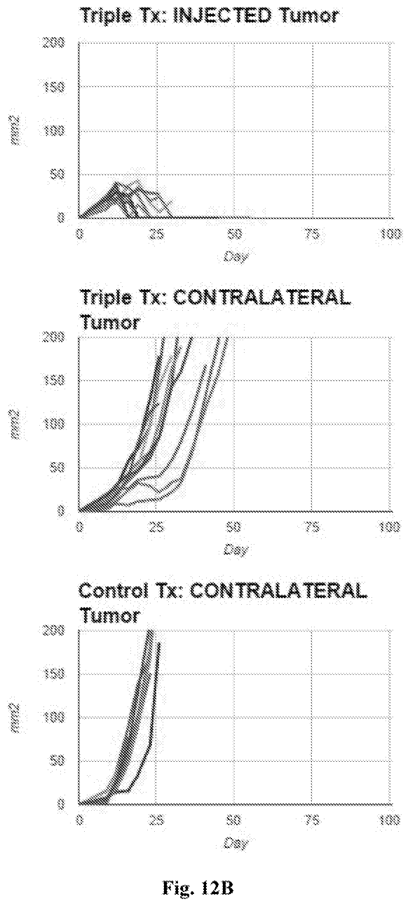

As shown in Example 1, injecting tumors with low-dose CD40 agonist mAb and MPL can synergize with intraperitoneal (IP) PD-1 mAb to treat cancer in an aggressive murine melanoma model, as well as other cancer models. The present Example extends upon the studies provided in Example 1 and provides data showing that intratumoral administration of a low-dose of CD40 agonist mAb and TLR4 agonist (MPL) can also synergize with intratumoral IL10R mAb—either alone or together with a PD-1 mAb—to treat cancer in the same B16 murine melanoma model. Experiments were performed to test the effects of intratumoral CD40 agonist mAb and intratumoral MPL in combination with either (A) IT IL10R mAb (FIG. 10 and FIG. 12), (B) IP PD-1 mAb (FIG. 9, and also experiments and Figures referred to in Example 1) or (C) both IL10R mAb and PD-1 mAb (FIG. 11). The same bilateral tumor model used above in Example 1 was used to test both the local and the systemic potency of these treatments.

In experiments similar to those described in Example 1, C57BL/6 mice were initially implanted with 5×10⁵ syngeneic B16F10 cells intradermally in bilateral flanks. 8 days post tumor cell implantation, when bilateral tumors measured ˜0.5 cm, intratumoral (IT) treatment with agents selected to activate APCs was initiated together with intraperitoneal (IP) anti-PD-1 mAb. Treatment was administered twice weekly for 4 weeks into one of the bilateral tumors. The contralateral tumor remained un-injected for the duration of the experiment (see FIG. 2 for schematic of experimental protocol). Various approaches reported to be involved in DC stimulation, such as activation of FLT3 and TLR3, were tested for their anti-tumor activity in the setting of either IP CTLA-4 or PD-1 blockade. The most potent treatment was the combination of the TLR4 agonist monophosphoryl lipid A (MPL) and low-dose CD40 agonist monoclonal antibody (mAb), both delivered IT—as shown in FIG. 9 (mice were treated with MPL (intratumoral) at 5 μg, anti-CD40 (intratumoral) at 20 μg, and anti-PD-1 (systemic via intraperitoneal injection) at 250 μg, while control mice were treated with isotype control antibodies and vehicle only).

In other similar experiments the combination of intratumoral (IT) MPL, IT CD40 agonist mAb, and IT IL10R blocking mAb was tested using the same experimental methodology as described above and shown in FIG. 2. The results are shown in FIG. 12. In this case the immunologic response at 24 hours after the initial treatment was also analyzed by flow cytometry. The results are shown in FIG. 10. These results confirm that DCs are indeed activated using this treatment method, as primarily evidenced by increased expression of CD86 on the DCs. Evidence of neutrophil expansion, Treg depletion, and gain of Helper T cell effector function was also noted—consistent with a strengthened immune response against the tumor.

Experiments were also performed using a combination of intratumoral MPL, intratumoral CD40 agonist mAb, intratumoral IL10 receptor blocking mAb, and systemic anti-PD-1 delivered intraperitoneally—once again using the same experimental system described in FIG. 2. The results are shown in FIG. 11.

Although the data presented here uses a melanoma model, the treatments and mechanisms of action are not cancer-type specific and, as illustrated in Example 1, are expected to translate to all cancer types.

### Example 3

The present Example relates to experiments similar to those provided in the preceding Examples, but that were performed utilizing nanoparticle technology to deliver an anti-CD40 antibody and MPL in physical association with one another and to test several different nanoparticles—i.e. those containing chitosan, chitosan with protamine, albumin, mannose, PLGA, or fucoidan. The anti-CD40 antibody was coated onto the surface of the nanoparticles and the TLR agonist MPL was included as cargo inside the nanoparticles. The anti-CD40/MPL nanoparticles were tested in the same bilateral mouse tumor models described in the previous Examples.

Nanoparticles were produced using ionotropic gelation such that each intratumoral injection delivered 5 μg MPL (as nanoparticle cargo) and 20 μg anti-CD40 mAb FGK45 (on the nanoparticle surface). These nanoparticles were administered to animals receiving 250 μg intraperitoneal anti-PD-1 (RMP1-14) concurrently. Non-formulated control animals received mixtures of MPL (5 μg) and FGK45 (20μg) injected intratumorally with RMP1-14 delivered intraperitoneally. Anti-PD-1-only control animals received 250 μg of intraperitoneal RMP1-14 alone. Isotype/vehicle control animals received isotype control mAbs and vehicle corresponding to the nanoparticle-treated group.

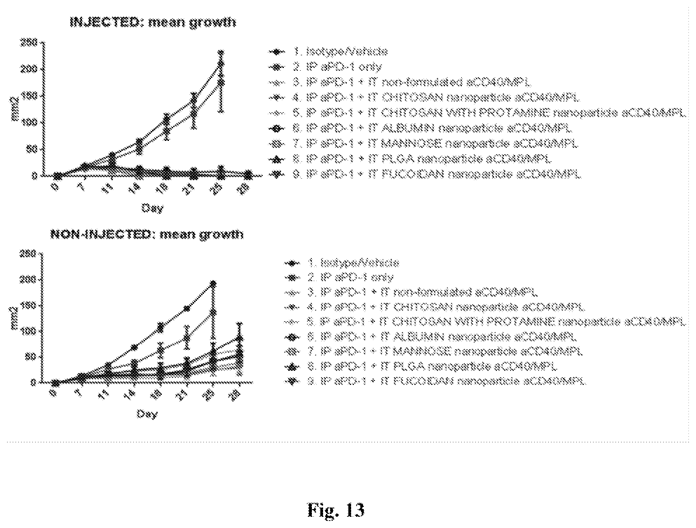

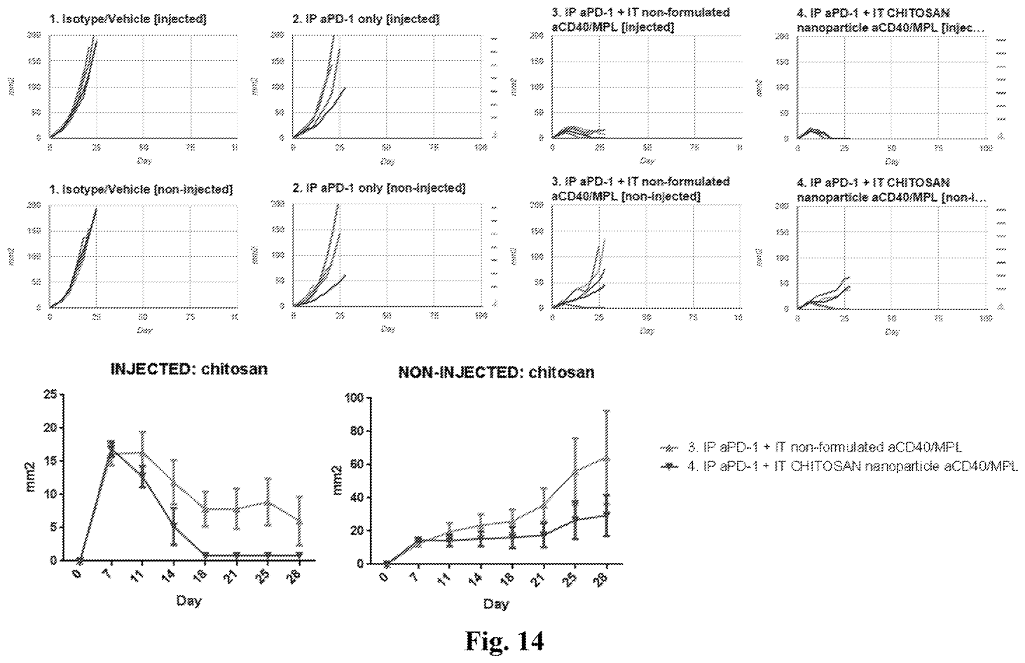

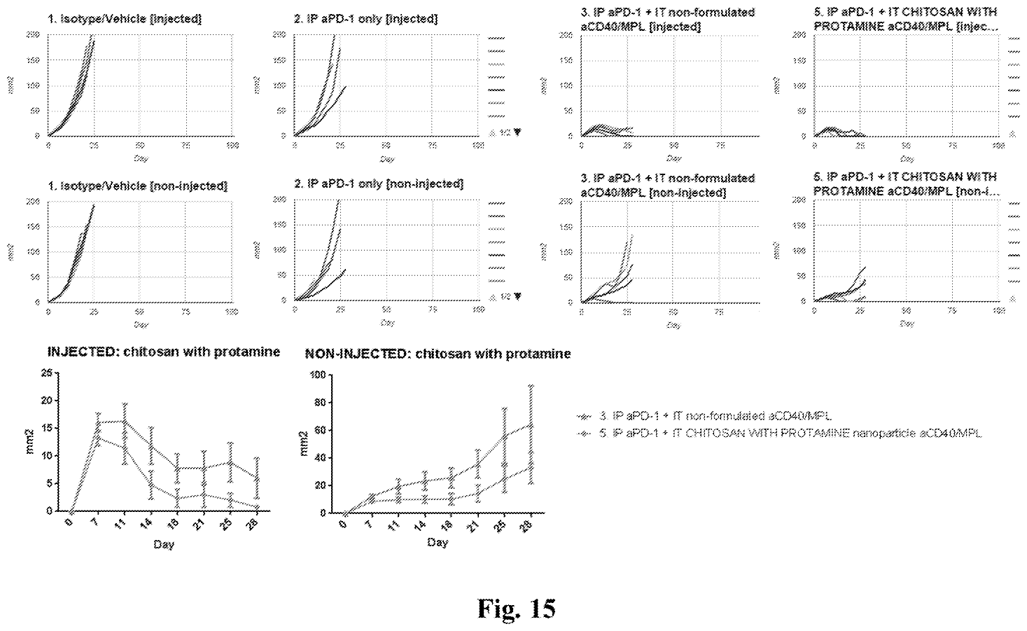

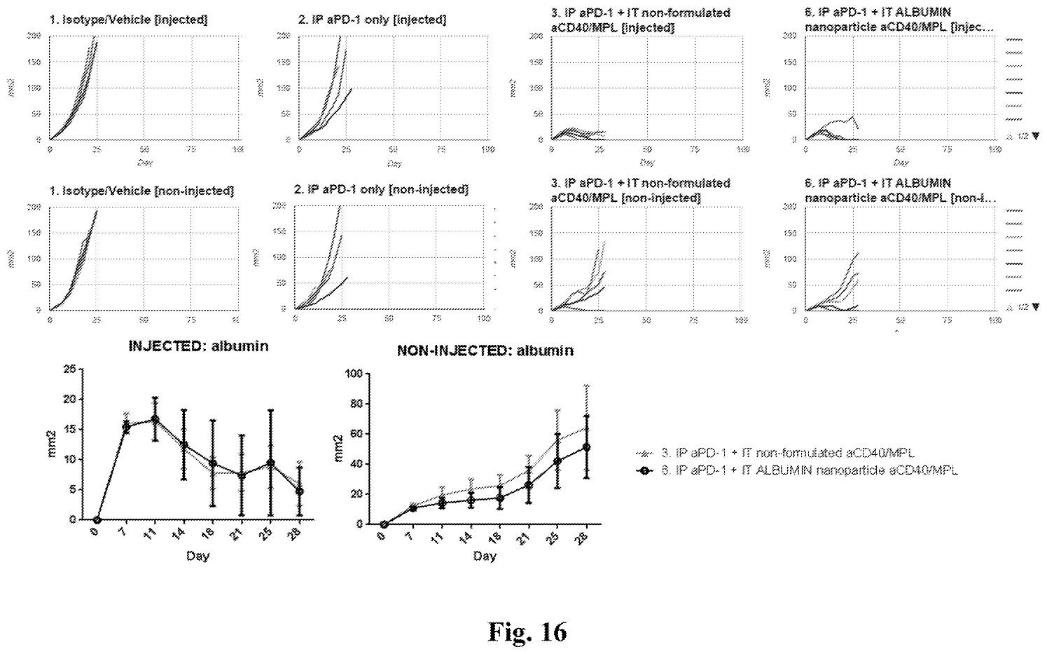

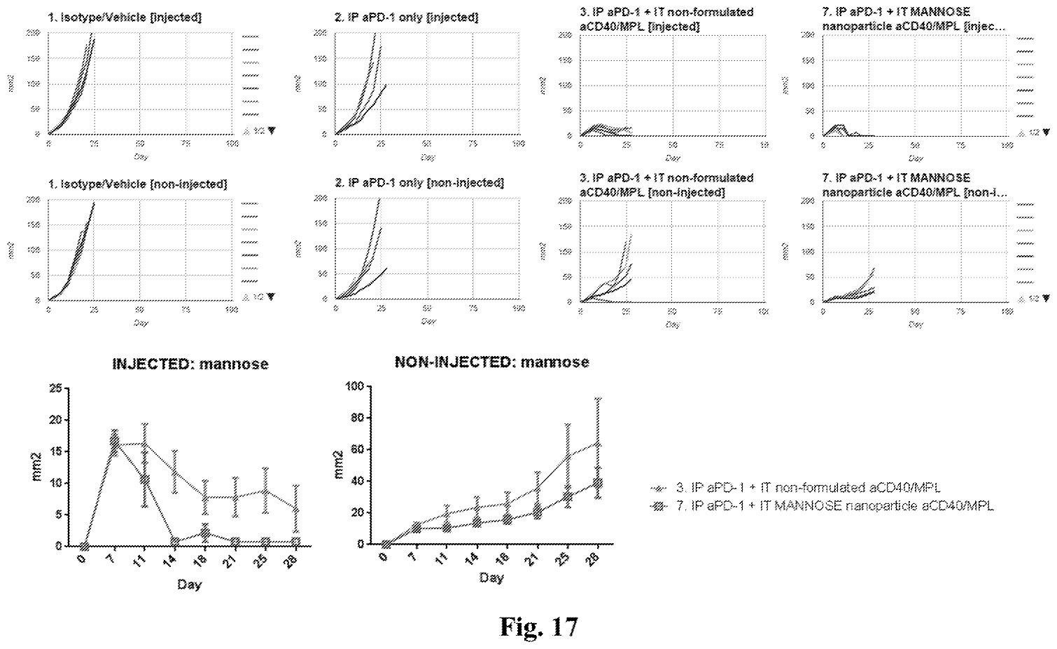

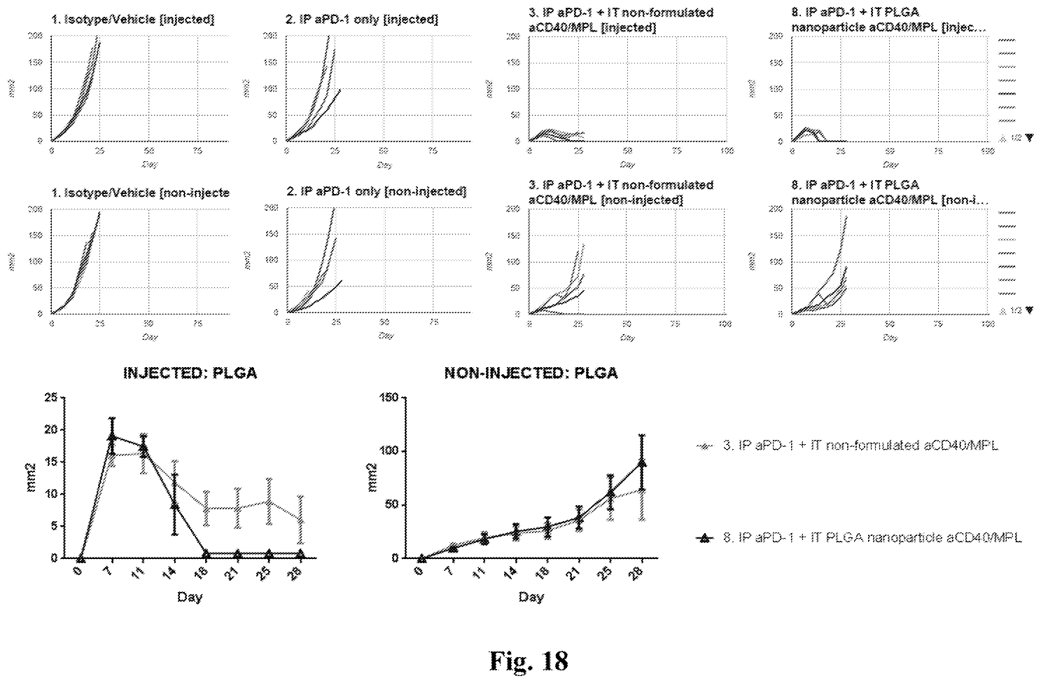

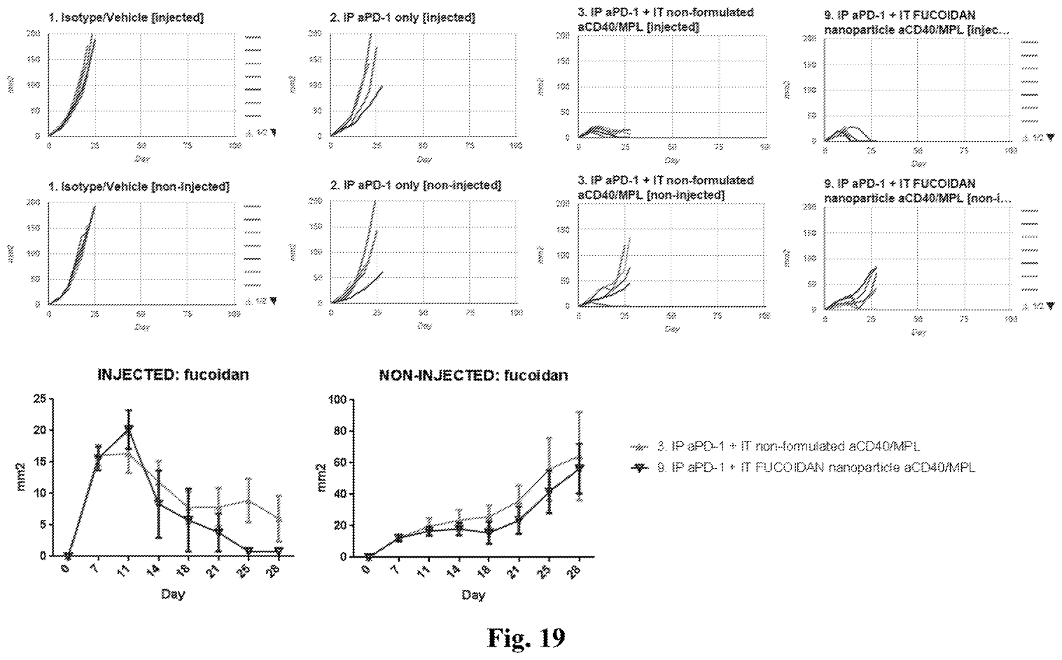

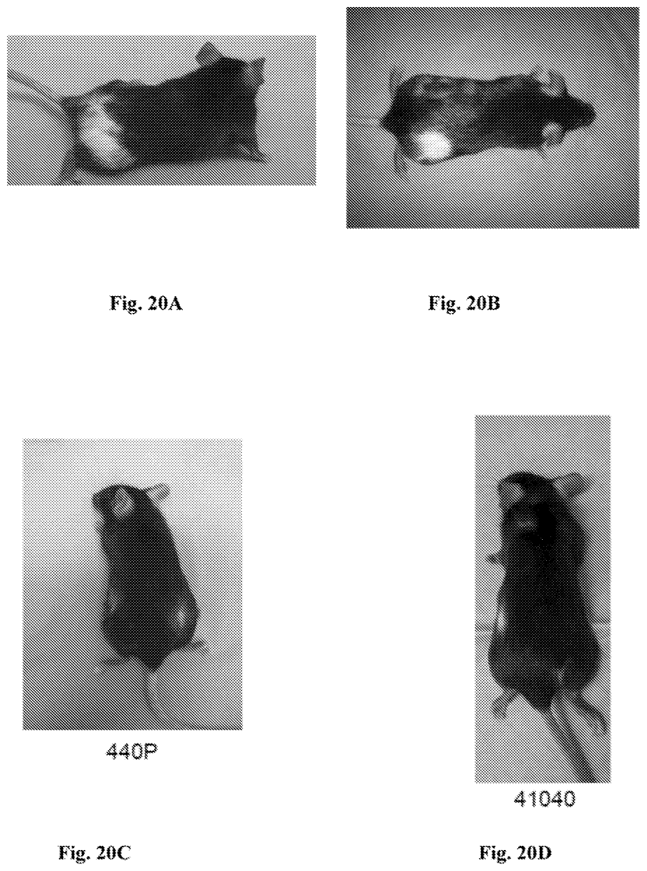

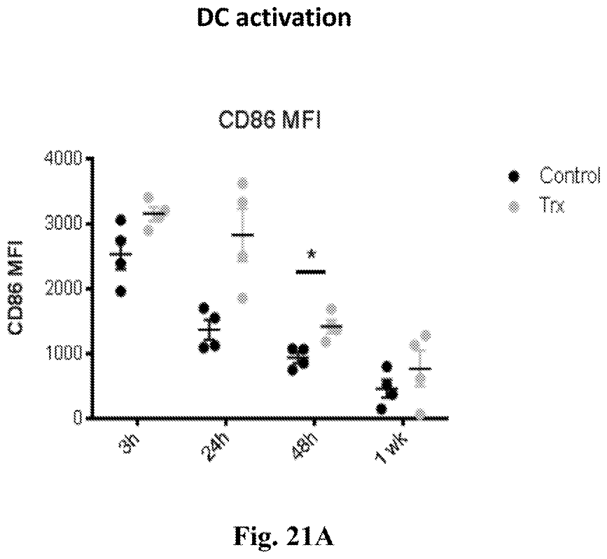

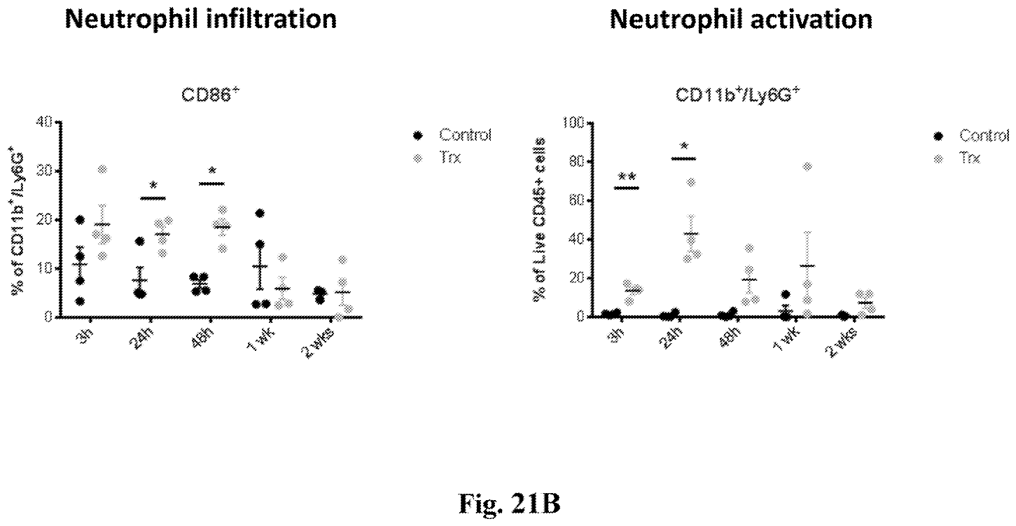

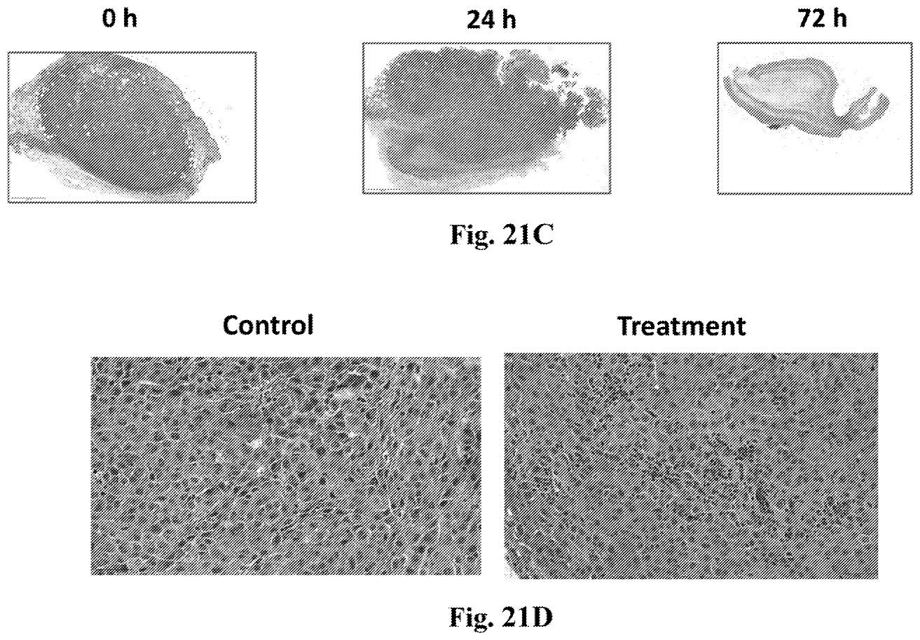

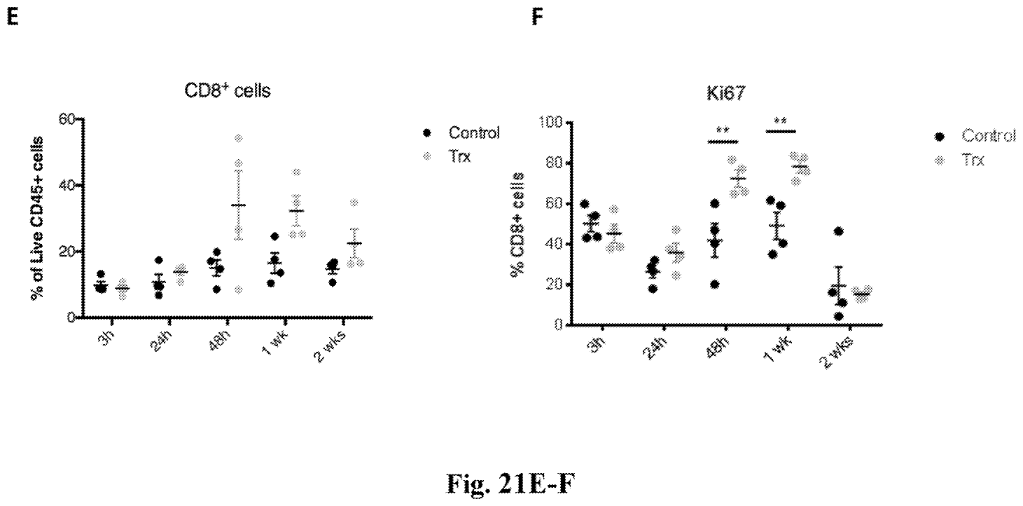

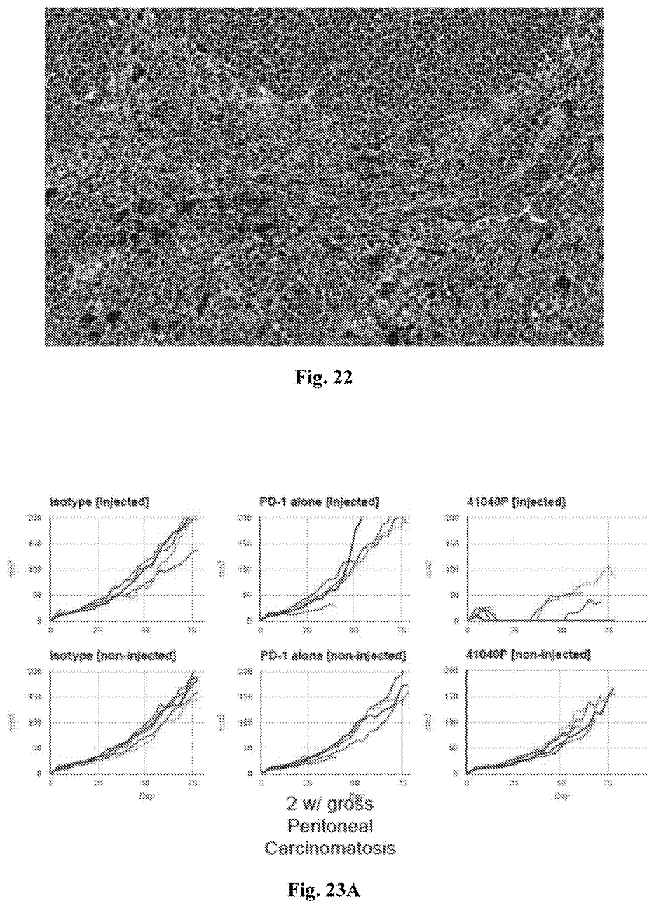

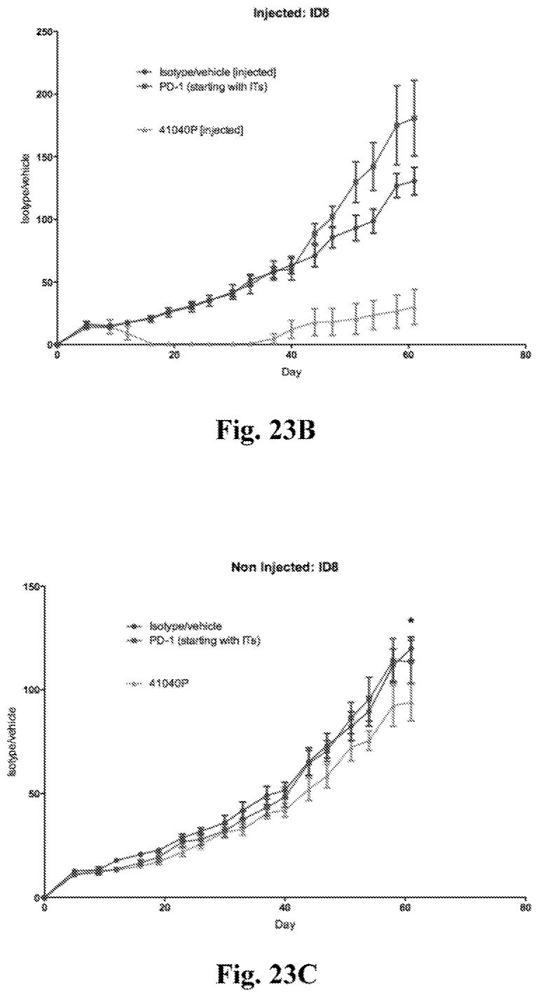

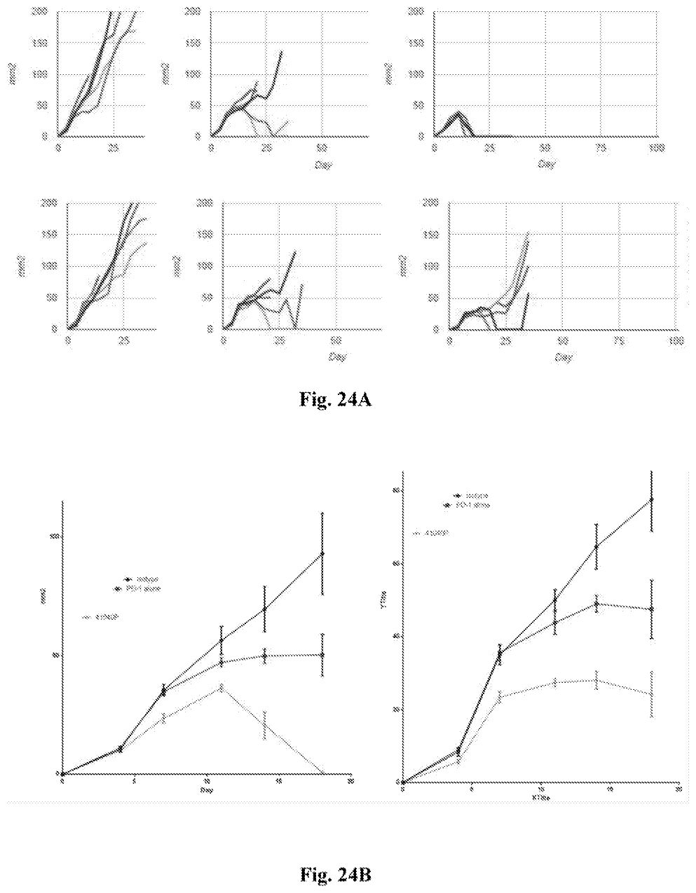

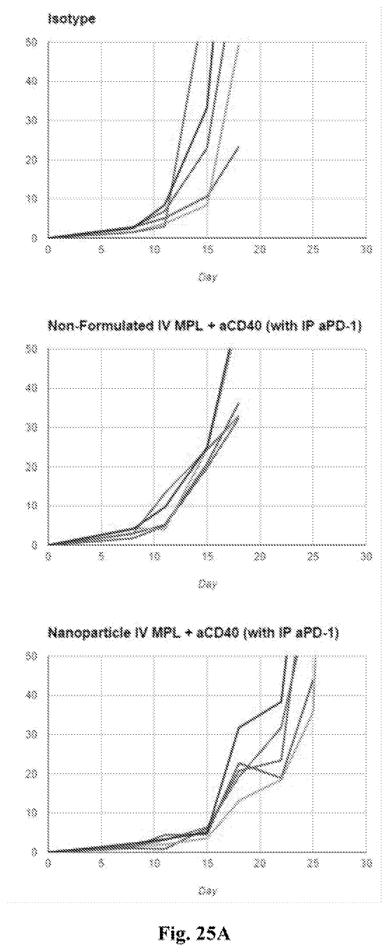

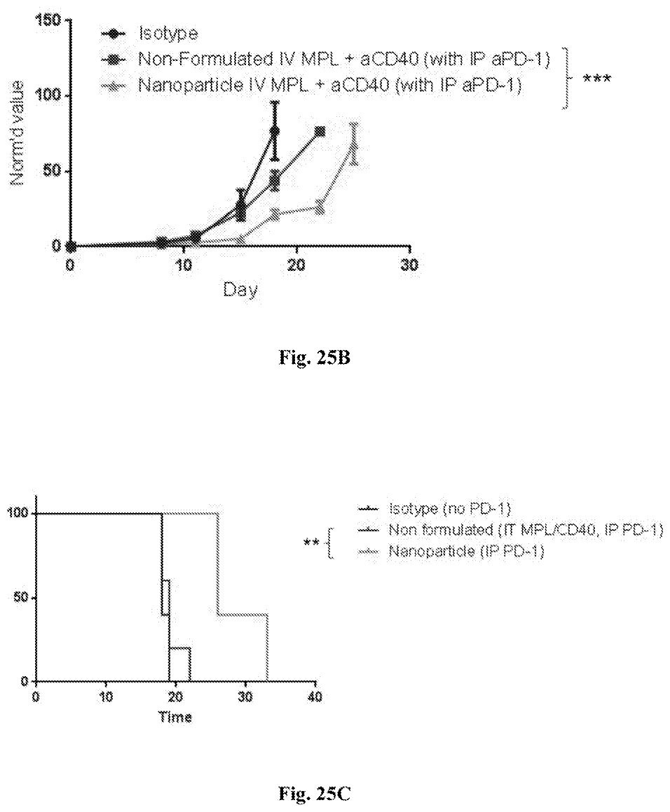

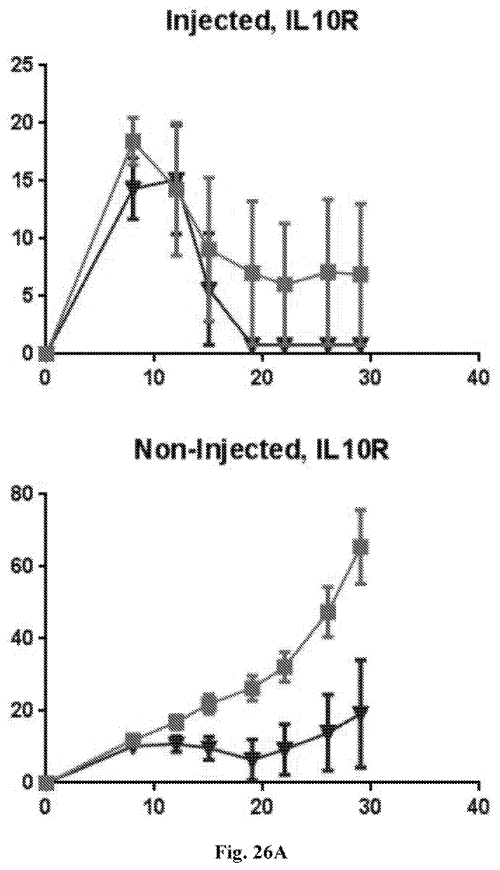

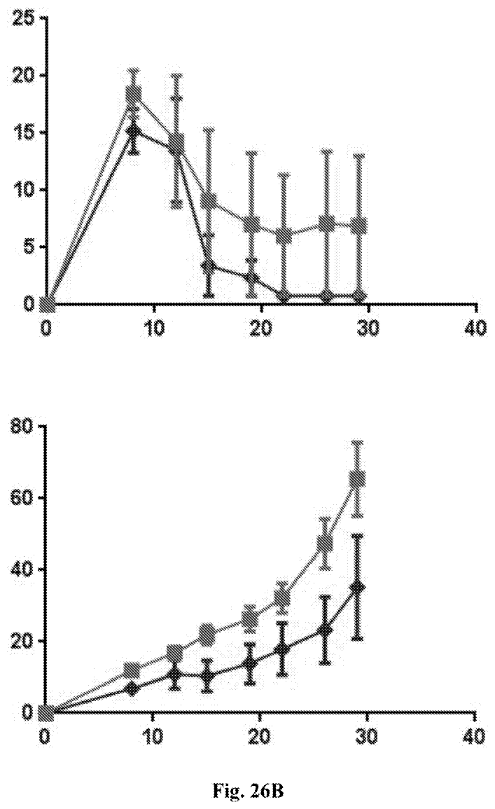

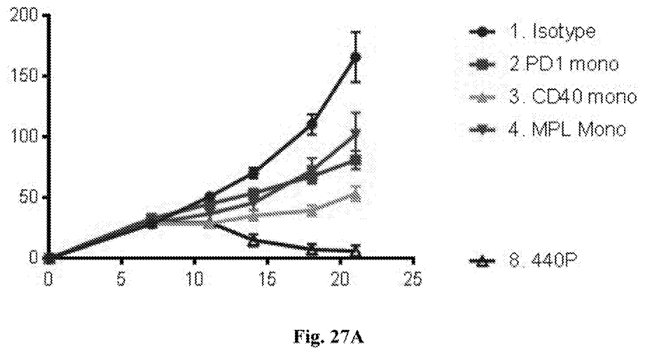

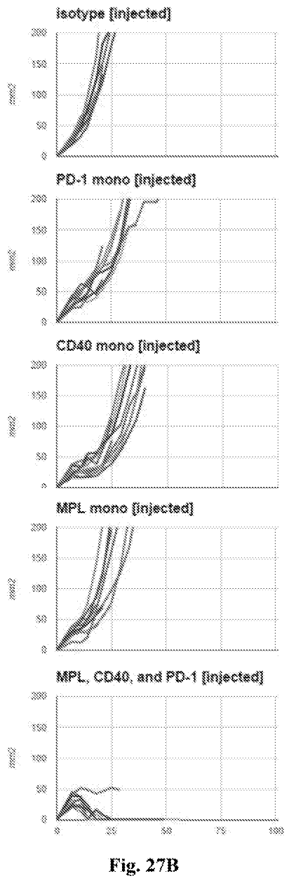

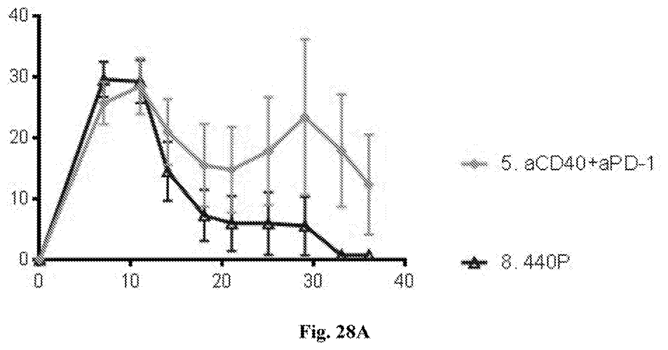

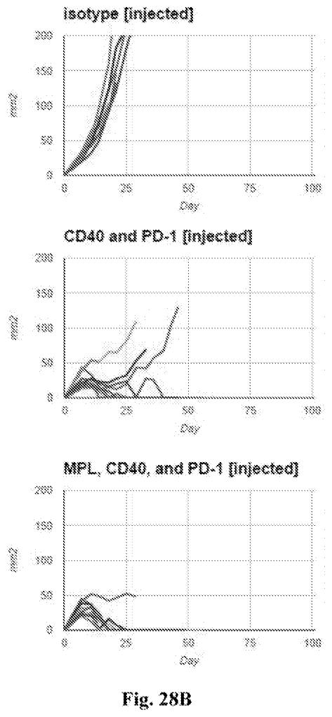

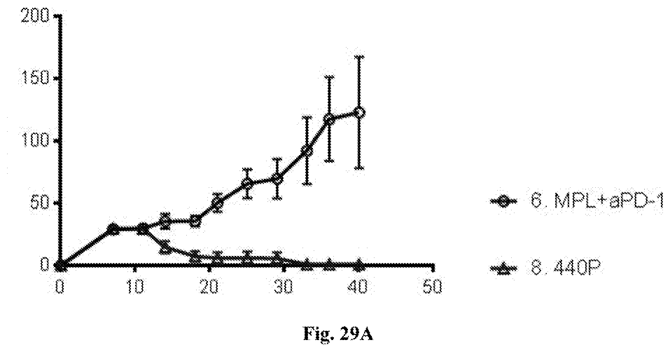

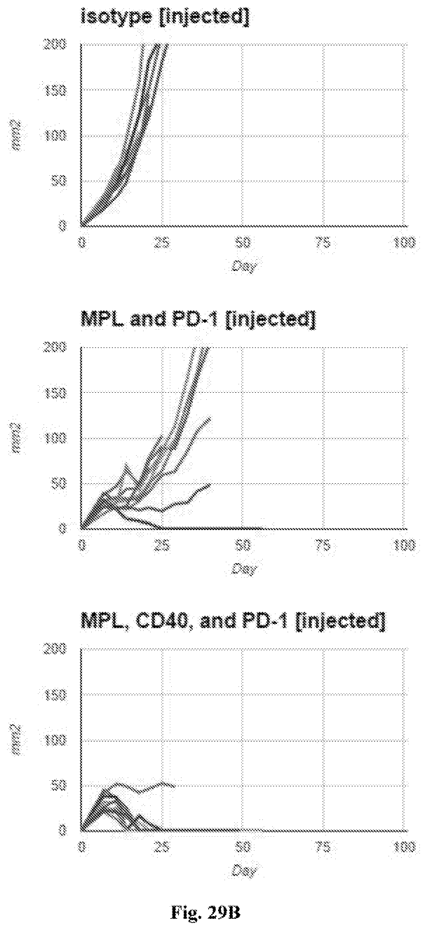

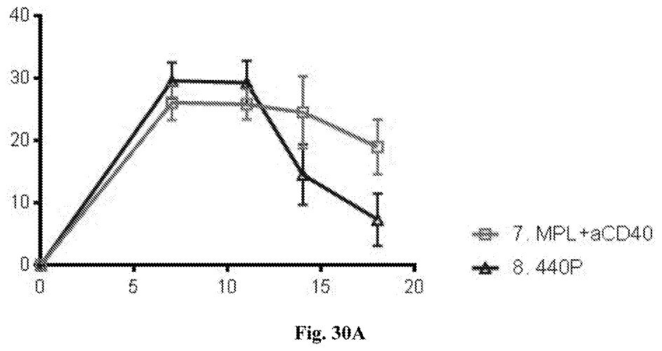

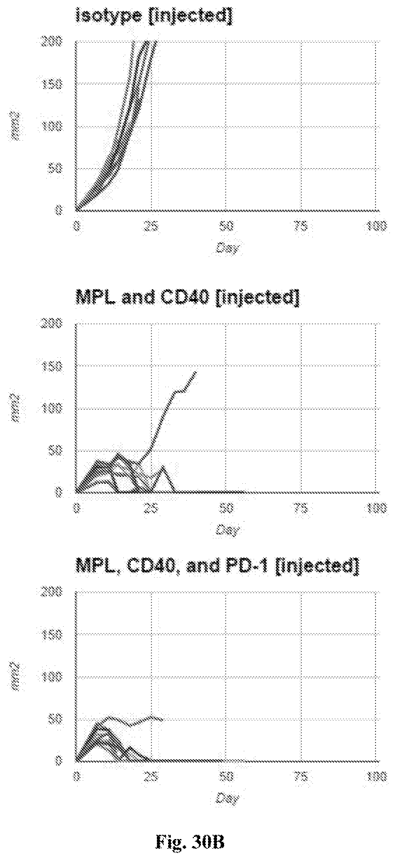

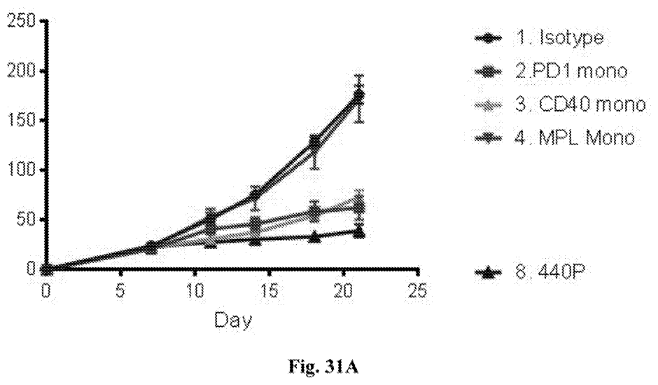

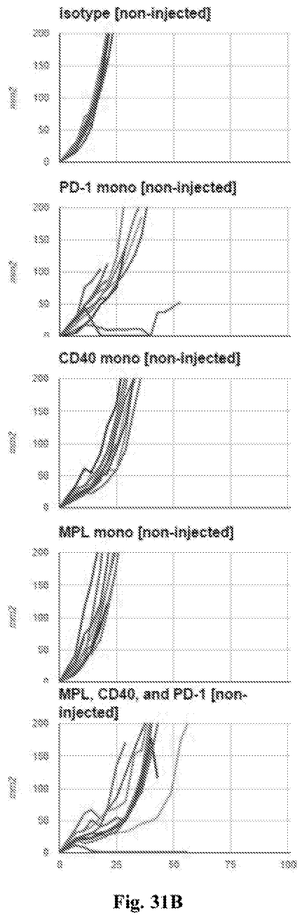

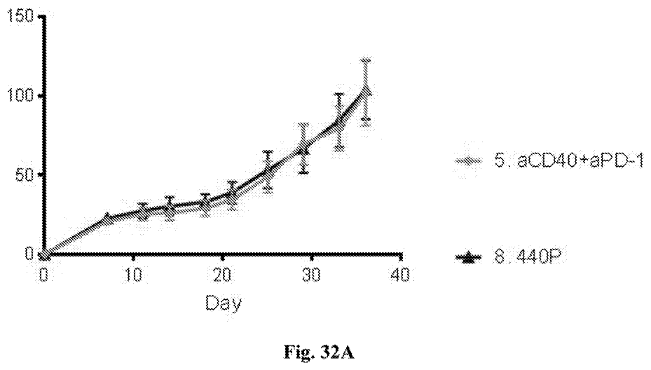

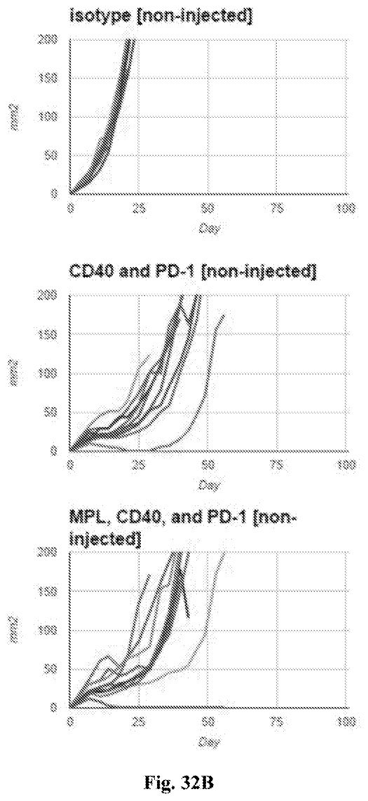

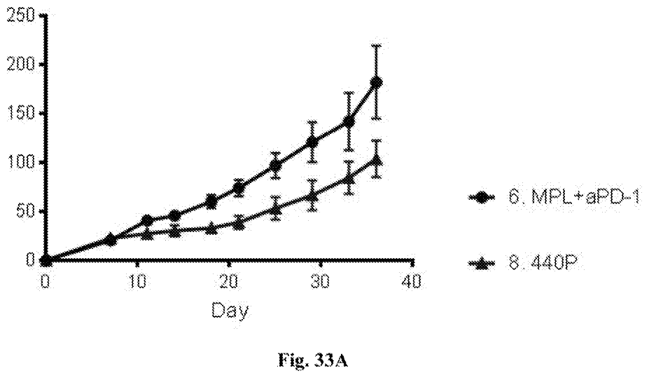

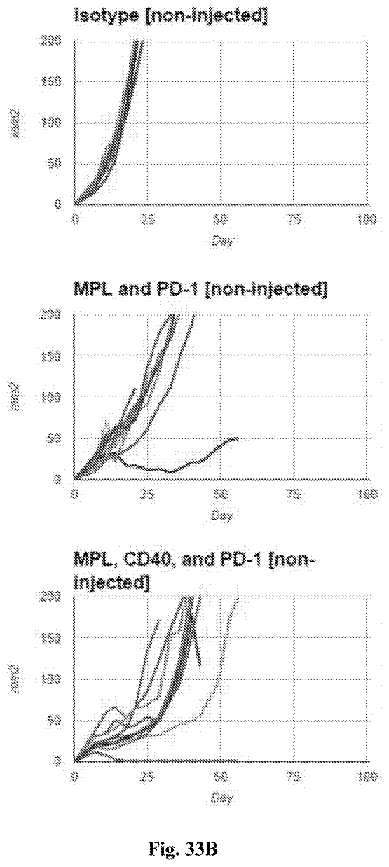

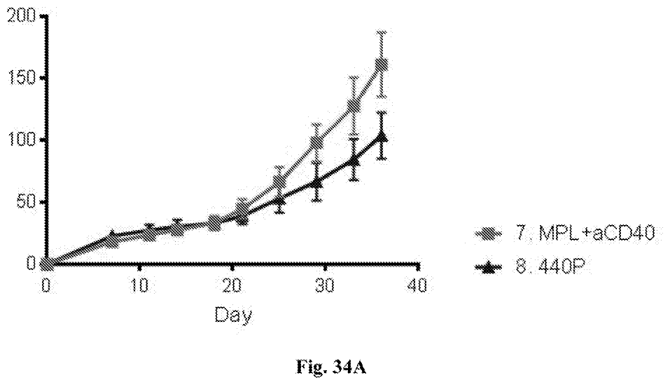

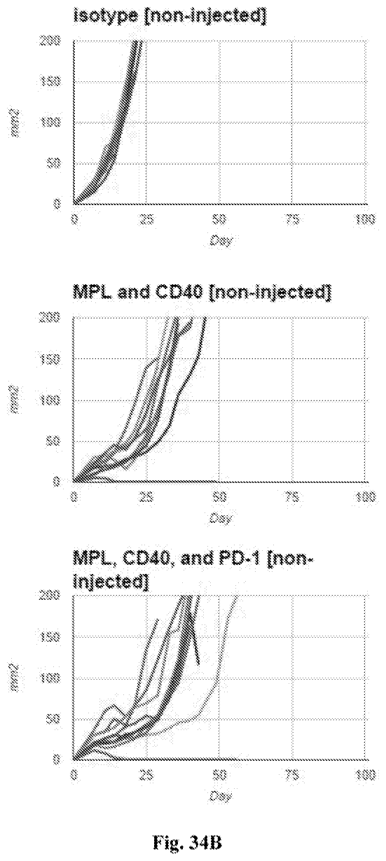

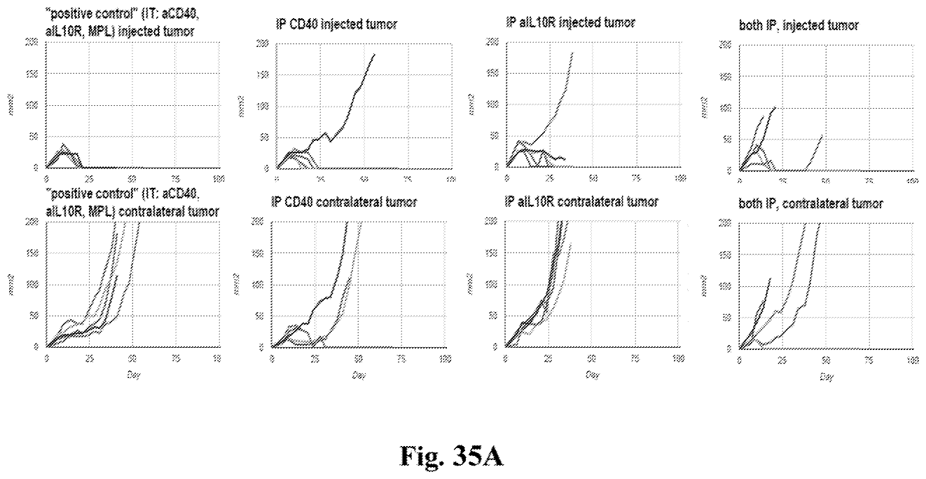

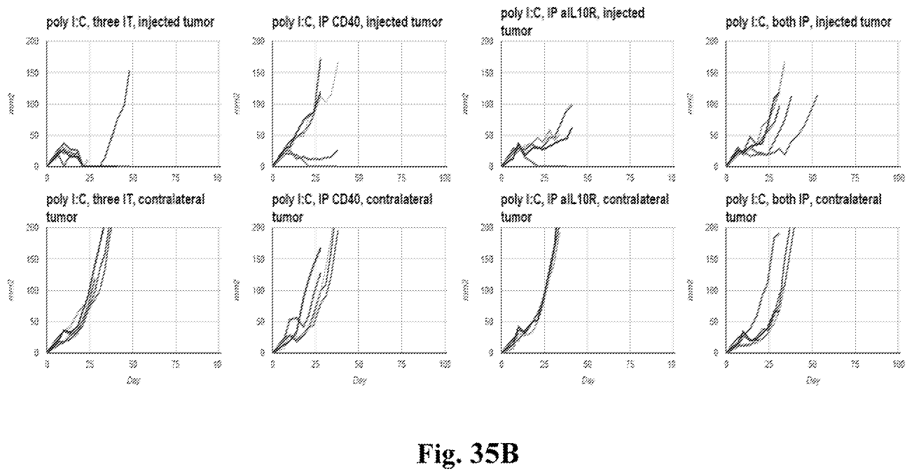

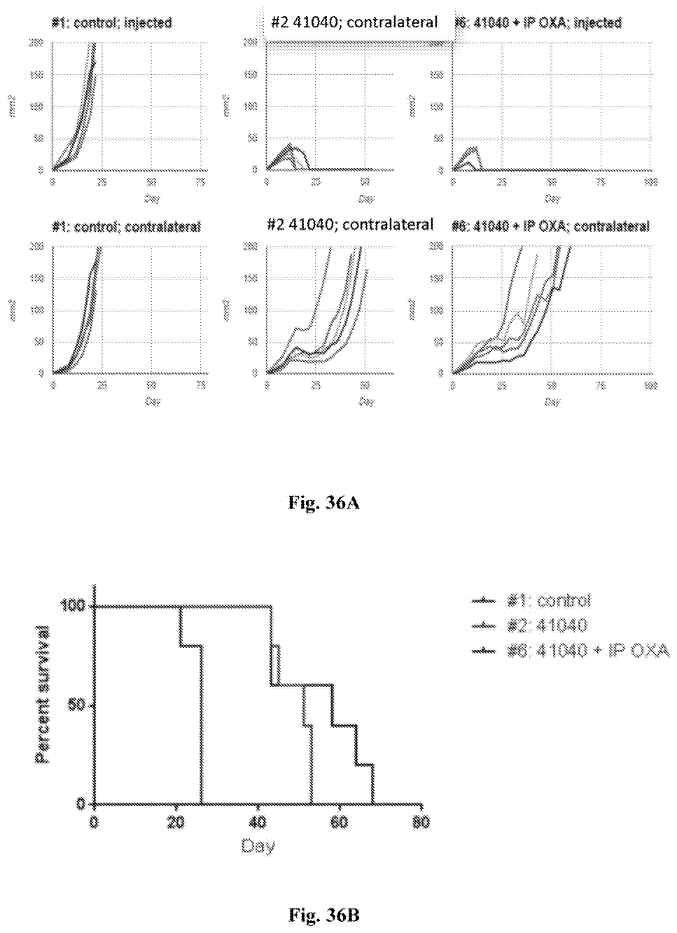

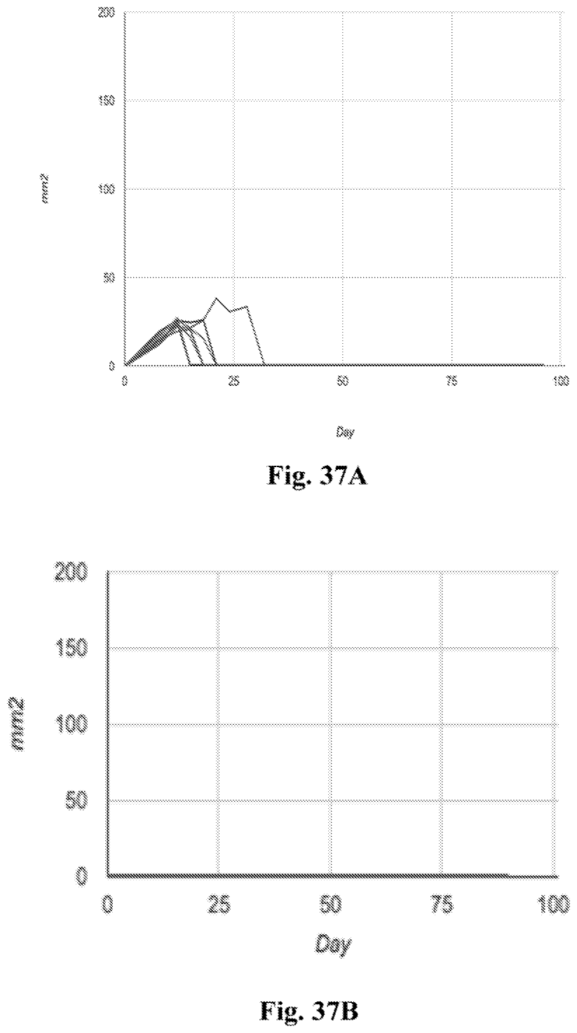

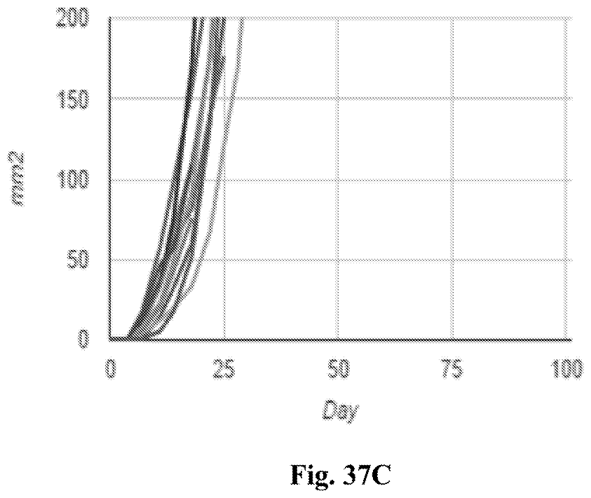

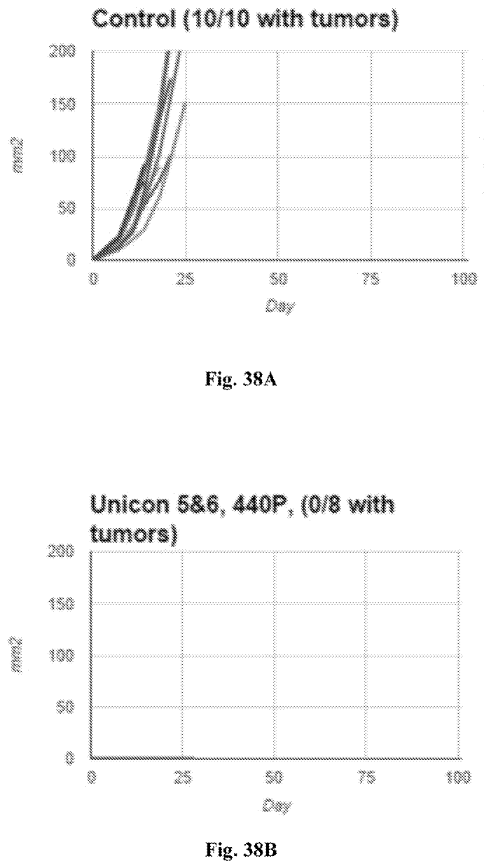

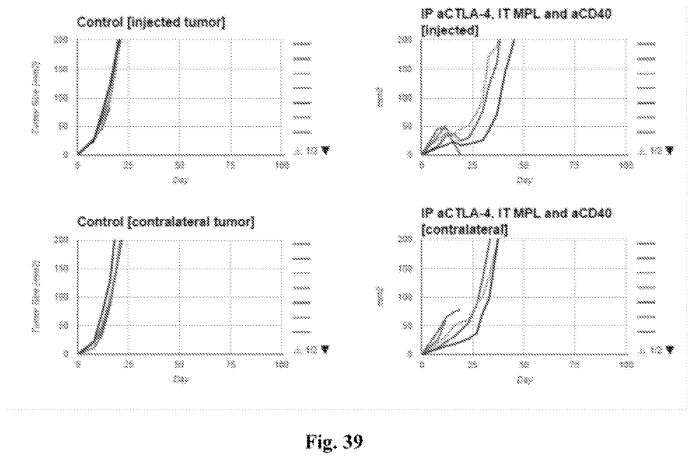

Results from such experiments are shown in FIGS. 13-19. These figure provide individual tumor growth curves (tumor size measured as surface area in mm²) over time (days) and/or averaged tumor growth curves from multiple tumors. The data indicates that most of the nanoparticle formulations provide superior local and/or distal tumor-control as compared to the non-formulated treatments.

### Example 4

Certain additional experiments were performed to expand upon the studies described above in the previous Examples, as follows:

Experiments were performed in which animals were treated with intratumoral MPL (5μg), the CD40 agonist mAb FGK45 (20μg), and intraperitoneal anti-PD-1 mAb RMP1-14 (250 μg) (see FIG. 20A and FIG. 20C), or with intratumoral MPL (5μg), FGK45 (20μg), and anti-IL10R mAb 1B1.3A (100μg) without PD-1 blockade (see FIG. 20B and FIG. 20D). As can be seen in FIG. 20A and FIG. 20B, animals developed a large patch of fur depigmentation at the site of intratumoral treatment for both treatments. At day 90, in the absence of ongoing treatment, animals were re-implanted with tumor cells in the contralateral flank. Animals resisted new tumor formation, and formed small patches of depigmented fur at the rechallenged site. See FIG. 20C and FIG. 20D. This data suggests the formation of antigen-specific, durable, immunologic memory capable of lysing tumor cells after treatment has ended. FIGS. 21A-C provide the results of further analysis of the regimen consisting of IT MPL, IT anti-CD40, and IP anti-PD-1, in which the data demonstrates that treatment with IT CD40 mAb, IT MPL, and systemic PD-1 causes rapid APC (e.g., DCs and neutrophils) accumulation and activation at the injected tumor. FIGS. 21D-F provide the results of further analysis performed with the same regimen showing that treatment with IT CD40 mAb, IT MPL, and systemic PD-1 causes subsequent infiltration and proliferation of CD8 T cells at the contralateral tumor.

Further experiments provided data showing that pigmented dendritic melanophages accumulate in the T-cell rich splenic peri-arterial lymphatic sheath 24 hours after a single treatment with intratumoral MPL (5μg), FGK45 (20μg), and anti-IL10R mAb 1B1.3A (100μg). See FIG. 22. These data suggest that melanoma-associated antigens are presented to T cells rapidly after treatment commences.

Further experiments were performed using the bilateral tumor model, referred to elsewhere herein, with different tumor cell lines. C57BL/6 animals were challenged bilaterally with ovarian carcinoma ID8 syngeneic tumor cells. Established tumors were treated with intratumoral MPL (5μg), FGK45 (20μg), and anti-IL10R mAb 1B1.3A (100μg) and intraperitoneal anti-PD-1 mAb RMP1-14 (250 μg). Control animals received either intraperitoneal RMP1-14 alone or isotype mAb in vehicle. These data demonstrate that intratumoral treatment with MPL, anti-CD40, and anti-IL10R with IP anti-PD-1 provides superior local and distal tumor control compared with the two control groups. The results of these experiments are provided in FIG. 23A-C. FIG. 24A-B shows data obtained from an experiment that was the same as that described above (for which the data is provided in FIG. 23) with the exception that syngeneic sarcoma LiHA tumor cells/tumors were used in place of syngeneic ovarian carcinoma ID8 tumor cells/tumors.

In additional experiments C57BL/6 animals were challenged intravenously (IV) with syngeneic HKP (krasG12D/+, p53f/f) lung carcinoma cells. Once bilateral lung tumors were established animals were treated once weekly for four weeks, and luminescence was assayed to monitor tumor growth. Animals received either isotype control mAbs, non-formulated mixtures of intravenous MPL (5 μg) and FGK45 (20 μg) together with 250 μg of RMP1-14 delivered intraperitoneally, or intravenous MPL (5 μg) and FGK45 (20 μg) formulated as a chitosan nanoparticle as described above together with 250 μg of RMP1-14 delivered intraperitoneally. The results of these experiments are shown in FIG. 25A-C. These data indicate that superior tumor control is achieved with the nanoparticle formulation. They also demonstrate the utility of such nanoparticles when administered intravenously.

Experiments were also performed to assess the effects of different surface antibodies and different cargo molecules in the context of the nanoparticles described herein. FIG. 26A-B provides data showing that the nanoparticle formulations described in the present patent application can be improved by adding either an anti-IL10R (1B1.3A) mAb to the surface or polyIC as cargo.

Experiments were also performed to assess the contribution of each agent in the regimen consisting of intratumoral MPL (5 μg per injection), intratumoral anti-CD40 mAb FGK45 (20 micro-grams per injection) and intraperitoneal anti-PD-1 mAb (250 μg per injection). The results of these experiments are presented in FIGS. 27-34. For the experiments, average tumor growth curves are presented (tumor surface area in mm² is shown on the Y axis plotted against time after tumor implantation in days on the X axis), as are individual tumor growth curves (tumor surface area in mm² is shown on the Y axis plotted against time after tumor implantation in days on the X axis). The data presented in FIGS. 27-34 shows the contribution of each single agent, and each doublet of agents, to the growth-control of the injected (FIGS. 27-30) and non-injected (FIGS. 31-34) tumors.

Experiments were performed to determine if anti-CD40 and/or anti-IL10R treatments could be effective systemically as well as intratumorally. As shown in FIG. 35A it was found that both agents, either alone or in combination, were indeed also effective when administered systemically.

Experiments were also performed to test whether other TLR agonists could be used in the treatment methods and compositions of the invention. As shown in FIG. 35B, it was found that PolyIC (a TLR3 agonist) could be substituted for MPL in such methods.

Experiments were also performed to test the effects of using the treatment methods and compositions of the present invention together with chemotherapy. As shown in FIG. 36A-B concurrent addition of systemic chemotherapy (in this case oxaliplatin or “OXA”) increased survival associated with intratumoral MPL, anti-IL10R, and anti-CD40 therapy in the bilateral mouse tumor model.

Experiments were also performed to investigate resistance of previously-treated mice to later tumor re-challenge. It was found that mice treated with a regimen of intratumoral MPL, anti-CD40, and anti-IL10R (which eradicated injected tumors as shown in FIG. 37A), resisted subsequent tumor re-challenge at day 90. As shown in FIG. 37B, 10/10 treated mice resisted tumor re-challenge, compared to 0/10 naïve controls (FIG. 37C). This effect was observed even though the doses of the active agents used in this experiments were halved as compared to the doses used in the other experiments described in the Examples section of this patent application (MPL was administered at 2.5 μg instead of 5 μg, anti-CD40 was administered at 10 μg instead of 20 μg, anti IL10R was administered at 50 μg instead of 100 μg). Similar results were obtained in mice treated with systemic anti-PD-1 together with intratumoral MPL and anti-CD40. See FIG. 38A-B. Thus, the treatment methods described herein result in the formation and persistence of anti-tumor immunologic memory.

Experiments were also performed to determine if the treatment regimens described herein could also be useful in the context of treatment with the immune checkpoint inhibitor anti-CTLA-4. FIG. 39 shows that a regimen of IT anti-CD40 mAb and MPL together with the immune checkpoint inhibitor anti-CTLA-4 (administered systemically via intraperitoneal injection) confers antitumor activity.

Experiments were also performed to determine if the anti-tumor activity observed at non-injected tumors using the methods and compositions of the invention is mediated by the adaptive immune system. If so, the antitumor activity should be lost in RAG1 knockout mice. Experiments were performed with control-treated animals, animals treated with systemic anti-PD-1, and intratumoral MPL and anti-CD40 (“440P”), animals treated with intratumoral MPL, anti-CD40, and anti-IL10R antibody (“41040”), and animals treated with systemic anti-PD-1, and intratumoral MPL, anti-CD40, and anti-IL10R (“41040P”). The anti-tumor activity manifested on non-injected tumors was virtually abolished in all treatment groups in the RAG knockout mice and survival was reduced—confirming that the effects are mediated by the adaptive immune system.

We claim:

1. A method of treating a tumor in a subject in need thereof, comprising administering to the subject an effective amount of a CD40 agonist antibody and a TLR agonist.

2. The method of claim 1, wherein the CD40 agonist antibody and the TLR agonist are administered locally, such as intratumorally.

3. The method of claim 1, further comprising administering to the subject an effective amount of an immune checkpoint inhibitor selected from the group consisting of a PD-1 inhibitor, a PD-L1 inhibitor, and a CTLA-4 inhibitor.

4. The method of claim 1, wherein the immune checkpoint inhibitor is administered systemically.

5. The method of claim 1 or claim 3, further comprising administering to the subject an effective amount of an IL10 receptor blocking antibody or an IL10 blocking antibody.

6. The method of claim 5, wherein the antibody is administered locally, such as intratumorally.

7. The method of claim 1, 3, or 5, wherein the subject has a tumor that is resistant to treatment with an immune checkpoint inhibitor.

8. The method of claim 1, 3, or 5, wherein the subject has a PD-1 or PD-L1 or CTLA-4 inhibitor resistant tumor.

9. The method of claim 1, 3, or 5, wherein the subject has previously been treated with a PD-1 or PD-L1 or CTLA-4 inhibitor.

10. The method of claim 1, 3, or 5, wherein the tumor is any solid tumor.

11. The method of claim 10, wherein the solid tumor is selected from the group consisting of a melanoma, a breast tumor, a lung tumor (such as a small cell lung cancer tumor), a prostate tumor, an ovarian tumor, a sarcoma, and a colon tumor.

12. The method of claim 1, 3, or 5, wherein the CD40 agonist antibody is a selected from the group consisting of FGK45, CP-870,984, CP-870,983, APX005M, dacetuzumab, ChiLob 7/4, a CD40 agonist antibody as described in WO2005/063289, and a CD40 agonist antibody as described in WO2013/034904.

13. The method of claim 1, 3, or 5, wherein the TLR agonist is any TLR agonist known in the art that binds to a TLR expressed by an antigen presenting cell (APC), such as a dendritic cell (DC), macrophages, tissue-resident macrophages, monocytes, monocyte-derived cells, B-Cells, neutrophils, langerhans cells, histiocytes, or any so-called professional or non-professional APC.

14. The method of claim 1, 3, or 5, wherein the TLR agonist is a TLR4 agonist.

15. The method of claim 14, wherein the TLR4 agonist is monophosphoryl lipid A (MPL).

16. The method of claim 1, 3, or 5, wherein the TLR agonist is a TLR3 agonist.

17. The method of claim 16, wherein the TLR3 agonist is polyI:C.

18. The method of claim 1, 3, or 5, wherein the TLR agonist is a TLR3 or TLR4 agonist.

19. The method of claim 1, 3, or 5, wherein the CD40 agonist antibody and the TLR agonist are connected via a linker moiety to form a single molecule.

20. The method of claim 19, wherein the linker is a lysine-bound linker or a cysteine-bound linker.

21. The method of claim 1, 3, or 5, comprising administering to the subject an effective amount of a nanoparticle comprising the CD40 agonist antibody and the TLR agonist.

22. The method of claim 5, comprising administering to the subject an effective amount of a nanoparticle comprising the CD40 agonist antibody, the TLR agonist, and the IL10 receptor blocking antibody or IL10 blocking antibody.

23. The method of claim 21 or 22, wherein the CD40 agonist antibody is present on the surface of the nanoparticles.

24. The method of claims 22, wherein the IL10 receptor blocking antibody or IL10 blocking antibody is present on the surface of the nanoparticles.

25. The method of claim 21 or 22, wherein the TLR agonist is present inside the nanoparticles.

26. The method of claim 21 or 22, wherein the nanoparticle comprises one or more agents selected from the group consisting of mannose, chitosan, manosylated chitosan, protamine, chitosan with protamine, albumin, PLGA, and fucoidan.

27. The method of claim 3 or 5, wherein the PD-1 inhibitor is an anti-PD1 antibody or the PD-L1 inhibitor is an anti-PD-L1 antibody or the CTLA-4 inhibitor is an anti-CTLA-4 antibody.

28. The method of claim 3 or 5, wherein the PD-1 inhibitor is the antibody RMP1-14.

29. The method of claim 5, wherein the IL10 receptor blocking antibody is the antibody 1B1.3A.

30. The method of claim 1, 3, or 5, wherein the CD40 agonist antibody is administered at a dose about 50 micrograms per intratumoral injection, or about 40 micrograms per intratumoral injection, or about 30 micrograms per intratumoral injection, or about 20 micrograms per intratumoral injection, or about 10 micrograms per intratumoral injection, or from about 10 micrograms to 50 micrograms per intratumoral injection.

31. The method of claim 1, 3, or 5, wherein the CD40 agonist antibody is administered at a dose that is less than 5% of the dose typically administered to a subject systemically for treatment of a tumor.

32. The method of claim 1, 3, or 5, wherein the CD40 agonist antibody is administered at a dose that is less than 4% of the dose typically administered to a subject systemically for treatment of a tumor.

33. The method of claim 1, 3, or 5, wherein the CD40 agonist antibody is administered at a dose that is less than 3% of the dose typically administered to a subject systemically for treatment of a tumor.

34. The method of claim 1, 3, or 5, wherein the CD40 agonist antibody is administered at a dose that is less than 2% of the dose typically administered to a subject systemically for treatment of a tumor.

35. The method of claim 1, 3, or 5, wherein the CD40 agonist antibody is administered at a dose that is less than 1% of the dose typically administered to a subject systemically for treatment of a tumor.

36. The method of claim 1, 3, or 5, wherein the TLR agonist is administered at a dose of about 25 micrograms per intratumoral injection, or about 20 micrograms per intratumoral injection, or about 15 micrograms per intratumoral injection, or about 10 micrograms per intratumoral injection, or about 5 micrograms per intratumoral injection, or less, or from about 1 microgram to about 25 micrograms per intratumoral injection.

37. The method of claim 3 or 5, wherein the PD-1 antibody, PD-L1 antibody, or CTLA-4 antibody, is administered at a dose of about 300 micrograms per IP injection, or about 250 micrograms per IP injection, or about 200 micrograms per IP injection, or about 150 micrograms per IP injection, or about 100 micrograms per IP injection.

38. The method of claim 5, wherein the IL10 receptor blocking antibody or IL10 blocking antibody is administered at a dose of about 200 micrograms per intratumoral injection, or about 150 micrograms per intratumoral injection, or about 100 micrograms per intratumoral injection, or about 80 micrograms per intratumoral injection, or about 60 micrograms per intratumoral injection, or about 50 micrograms per intratumoral injection, or about 40 micrograms per intratumoral injection, or about 20 micrograms per intratumoral injection, or less, or about 10 microgram to about 100 micrograms per intratumoral injection.

39. The method of claim 1, 3, or 5, wherein intratumoral APC maturation is stimulated in the subject.

40. The method of claim 1, 3, or 5, wherein intratumoral DC maturation is stimulated in the subject.

41. The method of claim 1, 3, or 5, wherein treatment results in regression of the injected tumor.

42. The method of claim 1, 3, or 5, wherein treatment results in regression of non-injected tumors.

43. A method of treating a tumor in a subject in need thereof, comprising administering to the subject an effective amount of: (a) a CD40 agonist antibody, (b) a TLR agonist, and (c) an IL10 receptor blocking antibody or an IL10 blocking antibody.

44. The method of claim 43, wherein each of the CD40 agonist antibody, the TLR agonist, and the IL10 receptor blocking antibody or IL10 blocking antibody are administered intratumorally.

45. The method of claim 44, wherein the subject has a tumor that is resistant to treatment with an immune checkpoint inhibitor.

46. The method of claim 44, wherein the subject has a PD-1, PD-L1, or CTLA-4 inhibitor resistant tumor.

47. The method of claim 44, wherein the subject has previously been treated with a PD-1 inhibitor a PD-L1 inhibitor or a CTLA-4 inhibitor.

48. The method of claim 44, wherein the tumor is any solid tumor.

49. The method of claim 48, wherein the solid tumor is selected from the group consisting of a melanoma, a breast tumor, a lung tumor (such as a small cell lung cancer tumor), a prostate tumor, an ovarian tumor, a sarcoma, and a colon tumor.

50. The method of claim 44, wherein the CD40 agonist antibody is a selected from the group consisting of f FGK45, CP-870,984, APX005M, dacetuzumab, ChiLob 7/4, a CD40 agonist antibody as described in WO2005/063289, and a CD40 agonist antibody as described in WO2013/034904.

51. The method of claim 44, wherein the TLR agonist is any TLR agonist known in the art that binds to a TLR expressed by an antigen presenting cell (APC), such as a dendritic cell (DC), macrophages, tissue-resident macrophages, monocytes, monocyte-derived cells, B-Cells, neutrophils, langerhans cells, histiocytes, or any so-called professional or non-professional APC.

52. The method of claim 44, wherein the TLR agonist is a TLR4 agonist.

53. The method of claim 52, wherein the TLR4 agonist is monophosphoryl lipid A (MPL).

54. The method of claim 44, wherein the TLR agonist is a TLR3 agonist.

55. The method of claim 54, wherein the TLR3 agonist is polyI:C.

56. The method of claim 44, wherein the TLR agonist is a TLR3 and/or TLR4 agonist.

57. The method of claim 44, wherein the CD40 agonist antibody and the TLR agonist are connected via a linker moiety to form a single molecule.

58. The method of claim 57, wherein the linker is a lysine-bound linker or a cysteine-bound linker.

59. The method of claim 44, comprising administering to the subject an effective amount of a nanoparticle comprising the CD40 agonist antibody and the TLR agonist.

60. The method of claim 44, comprising administering to the subject an effective amount of a nanoparticle comprising the CD40 agonist antibody, the TLR agonist, and the IL10 receptor blocking antibody or IL10 blocking antibody.

61. The method of claim 59 or 60, wherein the CD40 agonist antibody is present on the surface of the nanoparticles.

62. The method of claim 60, wherein the IL10 receptor blocking antibody or IL10 blocking antibody is present on the surface of the nanoparticles.

63. The method of claim 59 or 60, wherein the TLR agonist is present inside the nanoparticles.

64. The method of claim 59 or 60, wherein the nanoparticle comprises one or more agents selected from the group consisting of mannose, chitosan, manosylated chitosan, protamine, chitosan with protamine, albumin, PLGA, and fucoidan.

65. The method of claim 44, wherein the IL10 receptor blocking antibody is the antibody 1B1.3A.

66. The method of claim 44, wherein the CD40 agonist antibody is administered at a dose of about 50 micrograms per intratumoral injection, or about 40 micrograms per intratumoral injection, or about 30 micrograms per intratumoral injection, or about 20 micrograms per intratumoral injection, or about 10 micrograms per intratumoral injection, or from about 10 micrograms to 50 micrograms per intratumoral injection.

67. The method of claim 44, wherein the CD40 agonist antibody is administered at a dose that is less than 5% of the dose typically administered to a subject systemically for treatment of a tumor.

68. The method of claim 44, wherein the CD40 agonist antibody is administered at a dose that is less than 4% of the dose typically administered to a subject systemically for treatment of a tumor.

69. The method of claim 44, wherein the CD40 agonist antibody is administered at a dose that is less than 3% of the dose typically administered to a subject systemically for treatment of a tumor.

70. The method of claim 44, wherein the CD40 agonist antibody is administered at a dose that is less than 2% of the dose typically administered to a subject systemically for treatment of a tumor.

71. The method of claim 44, wherein the CD40 agonist antibody is administered at a dose that is less than 1% of the dose typically administered to a subject systemically for treatment of a tumor.

72. The method of claim 44, wherein the TLR agonist is administered at a dose of about 25 micrograms per intratumoral injection, or about 20 micrograms per intratumoral injection, or about 15 micrograms per intratumoral injection, or about 10 micrograms per intratumoral injection, or about 5 micrograms per intratumoral injection, or less, or from about 1 microgram to about 25 micrograms per intratumoral injection.

73. The method of claim 44, wherein the IL10 receptor blocking antibody or IL10 blocking antibody is administered at a dose of about 200 micrograms per intratumoral injection, or about 150 micrograms per intratumoral injection, or about 100 micrograms per intratumoral injection, or about 80 micrograms per intratumoral injection, or about 60 micrograms per intratumoral injection, or about 50 micrograms per intratumoral injection, or about 40 micrograms per intratumoral injection, or about 20 micrograms per intratumoral injection, or less, or about 10 microgram to about 100 micrograms per intratumoral injection.

74. The method of claim 44, wherein intratumoral APC maturation is stimulated in the subject.

75. The method of claim 44, wherein intratumoral DC maturation is stimulated in the subject.

76. The method of claim 44, wherein treatment results in regression of the injected tumor.

77. The method of claim 44, wherein treatment results in regression of non-injected tumors.

78. An antibody-drug conjugate molecule comprising: a CD40 agonist antibody and a TLR agonist linked via a linker moiety.

79. The antibody-drug conjugate molecule of claim 78, wherein the linker is a lysine-bound linker or a cysteine-bound linker.

80. The molecule of claim 78, wherein the CD40 agonist antibody is selected from the group consisting of f FGK45, CP-870,984, APX005M, dacetuzumab, ChiLob 7/4, a CD40 agonist antibody as described in WO2005/063289, and a CD40 agonist antibody as described in WO2013/034904.

81. The molecule of claim 78, wherein the TLR agonist is any TLR agonist known in the art that binds to a TLR expressed by an antigen presenting cell (APC), such as a dendritic cell (DC), macrophages, tissue-resident macrophages, monocytes, monocyte-derived cells, B-Cells, neutrophils, langerhans cells, histiocytes, or any so-called professional or non-professional APC.

82. The molecule of claim 78, wherein the TLR agonist is a TLR4 agonist.

83. The molecule of claim 82, wherein the TLR4 agonist is monophosphoryl lipid A (MPL).

84. The molecule of claim 78, wherein the TLR agonist is a TLR3 agonist.

85. The molecule of claim 84, wherein the TLR3 agonist is polyI:C.

86. The molecule of claim 78, wherein the TLR agonist is a TLR3 or TLR4 agonist.

87. A method of treating a tumor is a subject in need thereof, comprising administering to the subject an effective amount of the molecule of any one of claims 78-86.

88. The method of claim 87, wherein the molecule is administered intratumorally.

89. The method of claim 87, further comprising administering the subject an effective amount of a PD-1 inhibitor or a PD-L1 inhibitor or a CTLA-4 inhibitor.

90. The method of claim 89, wherein the PD-1 inhibitor or PD-L1 inhibitor or CTLA-4 inhibitor is administered systemically.

91. Use of a molecule according to any one of claims 78-86 in a method of treating a tumor in a subject in need thereof.

92. A pharmaceutical composition comprising the molecule of any one of claims 78-86.

93. A method of treating a tumor is a subject in need thereof, comprising administering to the subject an effective amount of the pharmaceutical composition of claim 92.

94. The method of claim 93, wherein the pharmaceutical composition is administered intratumorally.

95. The method of claim 93, further comprising administering the subject an effective amount of a PD-1 inhibitor or a PD-L1 inhibitor or a CTLA-4 inhibitor.

96. The method of claim 95, wherein the PD-1 inhibitor or PD-L1 inhibitor or CTLA-4 inhibitor is administered systemically.

97. Use of a pharmaceutical composition according to claim 92 in a method of treating a tumor in a subject in need thereof.

98. A pharmaceutical composition comprising: (a) a CD40 agonist antibody, and (b) a TLR agonist.

99. The pharmaceutical composition of claim 98, wherein the composition comprises one or more nanoparticles comprising both the CD40 agonist antibody and the TLR agonist.

100. The pharmaceutical composition of claim 99, wherein the CD40 agonist antibody is present on the surface of the nanoparticles.

101. The pharmaceutical composition of claim 99, wherein the TLR agonist is present inside the nanoparticles.

102. The pharmaceutical composition of claim 99, wherein the nanoparticle comprises one or more agents selected from the group consisting of mannose, chitosan, manosylated chitosan, protamine, chitosan with protamine, albumin, PLGA, and fucoidan.

103. A pharmaceutical composition comprising: (a) a CD40 agonist antibody, (b) a TLR agonist, and (c) an IL10 receptor blocking antibody or IL10 blocking antibody.

104. The pharmaceutical composition of claim 103, wherein the composition comprises one or more nanoparticles comprising each of the CD40 agonist antibody, the TLR agonist, and the IL10 receptor blocking antibody or IL10 blocking antibody.

105. The pharmaceutical composition of claim 104, wherein the CD40 agonist antibody is present on the surface of the nanoparticles.

106. The pharmaceutical composition of claim 104, wherein the IL10 receptor blocking antibody or IL10 blocking antibody is present on the surface of the nanoparticles.

107. The pharmaceutical composition of claim 104, wherein the TLR agonist is present inside the nanoparticles.

108. The pharmaceutical composition of claim 104, wherein the nanoparticle comprises one or more agents selected from the group consisting of mannose, chitosan, manosylated chitosan, protamine, chitosan with protamine, albumin, PLGA, and fucoidan.

109. A pharmaceutical composition comprising: (a) a CD40 agonist antibody, (b) a TLR agonist, and (c) a PD-1 inhibitor or PD-L1 inhibitor or CTLA-4 inhibitor.

110. The pharmaceutical composition of claim 109, wherein the composition comprises one or more nanoparticles comprising each of the CD40 agonist antibody, the TLR agonist, and the PD-1 inhibitor or PD-L1 inhibitor or CTLA-4 inhibitor.

111. The pharmaceutical composition of claim 110, wherein the CD40 agonist antibody is present on the surface of the nanoparticles.

112. The pharmaceutical composition of claim 110, wherein the TLR agonist is present inside the nanoparticles.

113. The pharmaceutical composition of any claim 110, wherein the PD-1 inhibitor or PD-L1 inhibitor or CTLA-4 inhibitor is present inside the nanoparticles.

114. The pharmaceutical composition of claim 110, wherein the nanoparticle comprises one or more agents selected from the group consisting of mannose, chitosan, manosylated chitosan, protamine, chitosan with protamine, albumin, PLGA, and fucoidan.

115. A pharmaceutical composition comprising: (a) a CD40 agonist antibody, (b) a TLR agonist, a (c) a PD-1 inhibitor or PD-L1 inhibitor or CTLA-4 inhibitor, and (d) an IL10 receptor blocking antibody or IL10 blocking antibody.

116. The pharmaceutical composition of claim 115, wherein the composition comprises one or more nanoparticles comprising each of the CD40 agonist antibody, the TLR agonist, the PD-1 inhibitor or PD-L1 inhibitor or CTLA-4 inhibitor, and the IL10 receptor blocking antibody or IL10 blocking antibody.

117. The pharmaceutical composition of claim 116, wherein the CD40 agonist antibody is present on the surface of the nanoparticles.

118. The pharmaceutical composition of claim 116, wherein the IL10 receptor blocking antibody or IL10 blocking antibody is present on the surface of the nanoparticles.

119. The pharmaceutical composition of claim 116, wherein the TLR agonist is present inside the nanoparticles.

120. The pharmaceutical composition of claim 116, wherein the PD-1 inhibitor or PD-L1 inhibitor or CTLA-4 inhibitor is present inside the nanoparticles.

121. The pharmaceutical composition of claim 116, wherein the nanoparticle comprises one or more agents selected from the group consisting of mannose, chitosan, manosylated chitosan, protamine, chitosan with protamine, albumin, PLGA, and fucoidan.

122. The pharmaceutical composition of any one of claims 98-121, wherein the CD40 agonist antibody is selected from the group consisting of FGK45, CP-870,984, APX005M, dacetuzumab, ChiLob 7/4, a CD40 agonist antibody as described in WO2005/063289, and a CD40 agonist antibody as described in WO2013/034904

123. The pharmaceutical composition of any one of claims 98-121, wherein the TLR agonist is any TLR agonist known in the art that binds to a TLR expressed by an antigen presenting cell (APC), such as a dendritic cell (DC), macrophages, tissue-resident macrophages, monocytes, monocyte-derived cells, B-Cells, neutrophils, langerhans cells, histiocytes, or any so-called professional or non-professional APC.

124. The pharmaceutical composition of any one of claims 98-121, wherein the TLR agonist is a TLR4 agonist.

125. The pharmaceutical composition of claim 124, wherein the TLR4 agonist is monophosphoryl lipid A (MPL).

126. The pharmaceutical composition of any one of claims 98-124, wherein the TLR agonist is a TLR3 agonist.

127. The pharmaceutical composition of claim 126, wherein the TLR3 agonist is polyI:C.

128. A method of treating a tumor in a subject in need thereof, comprising administering to the subject an effective amount of the pharmaceutical composition of any one of claims 98-127.

129. The method of claim 128, wherein the pharmaceutical composition is administered locally (such as intratumorally), or intravenously.

130. Use of a pharmaceutical composition according to any one of claims 98-127 in a method of treating a tumor in a subject in need thereof.
